(19) United States
(12) Patent Application Publication
(10) Pub. No.: US 2024/0247234 A1
(43) Pub. Date: Jul. 25, 2024

(54) OFF-THE-SHELF HUMAN UMBILICAL CORD-DERIVED MESENCHYMAL STEM CELLS AND PREPARATION METHOD AND USE THEREOF

(71) Applicant: WUHAN OPTICS VALLEY ZHONGYUAN PHARMACEUTICAL CO., LTD., Wuhan (CN)

(72) Inventors: Yu ZHANG, Wuhan (CN); Qi QI, Wuhan (CN); Yun ZHANG, Wuhan (CN); Ruizhen HOU, Wuhan (CN); Wenjie WU, Wuhan (CN); Wangyi ZHOU, Wuhan (CN); Lei WANG, Wuhan (CN)

(21) Appl. No.: 18/401,238

(22) Filed: Dec. 29, 2023

Related U.S. Application Data
(63) Continuation of application No. PCT/CN2022/116699, filed on Sep. 2, 2022.

(30) Foreign Application Priority Data
Dec. 31, 2021 (CN) 202111672027.1

(51) Int. Cl. C12N 5/0775; A61K 35/28; A61P 1/16; A61P 11/00; A61P 31/14

(52) U.S. Cl. CPC C12N 5/0665; A61K 35/28; A61P 1/16; A61P 11/00; A61P 31/14; C12N 2523/00

(57) ABSTRACT

TA preparation method for off-the-shelf human umbilical cord-derived mesenchymal stem cells includes culturing an isolated umbilical cord tissue blocks to obtain P0 cells; carrying out passage on the P0 cells twice to obtain P2 cells, and cryopreserving the P2 cells; the cryopreserved P2 cells are subjected to thawing and passage twice to obtain P4 cells, and the P4 cells are cryopreserved; the cryopreserved P4 cells are subjected to thawing and passage once to obtain P5 cells, and the P5 cells are sequentially digested, washed and resuspended to obtain the human umbilical cord-derived mesenchymal stem cells. The present invention also adopts cryopreservation method to develop a cryopreservation preparation of human umbilical cord-derived mesenchymal stem cells. The cryopreservation preparation is convenient for storage and can be directly administered intravenously after thawing without changing the liquid, avoiding possible contamination risk during dispensing, and is more conducive to clinical use.

### RELATED APPLICATIONS

The present application is a Continuation of International Application Number PCT/CN2022/116699 filed Sep. 2, 2022, which claims priority to Chinese Application Number 202111672027.1 filed Dec. 31, 2021, the disclosures of which are hereby incorporated by reference herein in their entireties.

### INCORPORATION BY REFERENCE

The sequence listing provided in the file entitled C6351-112 SQL.xml, which is an Extensible Markup Language (XML) file that was created on Mar. 15, 2024, and which comprises 6,495 bytes, is hereby incorporated by reference in its entirety.

### FIELD OF THE INVENTION

The present invention belongs to the field of biotechnology, and in particular relates to an off-the-shelf product of human umbilical cord-derived mesenchymal stem cells (hUC-MSCs) and preparation method and use thereof.

### BACKGROUND OF THE INVENTION

Mesenchymal stem cells (MSCs) are a group of multipotent adult stem cells derived from mesoderm, which have the potential of self-renewal and multi-directional differentiation, promoting tissue and organ repair and immune regulation, etc. MSC can be isolated and cultured from a variety of tissues such as bone marrow, umbilical cord, placenta, adipose tissue, bone, dental pulp and endometrium. Additionally, MSC-like cells can also be differentiated from embryonic stem cells or pluripotent stem cells. MSC has low immunogenicity and is less likely to cause immune rejection because it does not express costimulatory molecules such as CD40, CD80, CD86, and major histocompatibility complex (MHC) class II molecules. Meanwhile, it can exert immune regulatory effects on both autologous and allogeneic immune cells as its regulatory effect on immune cells is not limited by major histocompatibility complex molecules. With the inflammatory chemotactic properties, MSCs can migrate to inflammatory sites by sensing inflammatory signals (cytokines, chemokine receptors, integrins, etc.), and play the function relying on direct cell-to-cell contact and/or paracrine effects. For example, MSC can regulate the proliferation and differentiation of abnormally activated T lymphocytes, B lymphocytes, natural killer cells, dendritic cells, etc., as well as the production of antibodies through direct cells contact or cytokines secretion. A number of clinical studies proved that MSC infusion can significantly reduce the secretion of pro-inflammatory cytokines, IFN-γ, IL-2, IL-12 and IL-17A in patients. MSC-promoted secretion of IL-10 and TGF-ß can inhibit abnormally activated Th1 cells, restore Th1/Th2 balance, inhibit excessive proliferation of T cells, and inhibit the activity of cytotoxic CD8+ T lymphocytes through the NKG2D pathway. MSC can also repolarize macrophages from a pro-inflammatory M1 phenotype to an anti-inflammatory M2 phenotype in an LPS-dependent manner through the synergistic action of their own secreted IL-10 and TGF-β cytokines. Studies confirmed that the white blood cell count and neutrophil count of patients treated with MSC decreased to normal levels, and the counts of CD3+ T cells, CD4+ T cells and CD8+ T cells increased to normal levels.

At present, MSCs have been widely used in clinical application to treat a variety of diseases. The following MSC drugs or MSC-based therapies have been approved worldwide: 1. Queencell, derived from autologous adipose tissue, approved in South Korea in 2010 for the indication of subcutaneous tissue defects; 2. Cellgram-AMI, derived from autologous bone marrow, approved in South Korea in 2011 for the indication of acute myocardial infarction; 3. Cartistem, derived from allogeneic cord blood, approved in South Korea in 2012 for the indication of knee cartilage defect; 4. Cupistem, derived from autologous adipose tissue, approved in South Korea in 2012 for the indication of Crohn's disease complex anal fistula; 5. Prochymal, derived from allogeneic bone marrow, approved in New Zealand in 2012 for the indication of acute graft-versus-host disease (children); 6. Neronata-R, derived from autologous bone marrow, approved in South Korea in 2014 for the indication of amyotrophic lateral sclerosis; 7. Prochymal, derived from allogeneic bone marrow, approved in Canada in 2015 for the indication of acute graft-versus-host disease (children); 8. Temcell, derived from allogeneic bone marrow, approved in Japan in 2015 for the indication of acute graft-versus-host disease; 9. Alofisel, derived from allogeneic adipose tissue, approved in the EU in 2018 for the indication of Crohn's disease complex anal fistula; 10. Stemirac, derived from allogeneic bone marrow, approved in Japan in 2018 for the indication of spinal cord injury; 11. Stempeucel, derived from allogeneic adipose tissue, approved in India in 2020 for the indication of severe limb ischemia; 12. Alofisel, derived from allogeneic adipose tissue, approved in Japan in 2021 for the indication of Crohn's disease complex anal fistula. It can be seen that the stem cells used in the current MSC-based therapies are usually derived from bone marrow or adipose tissue, and no MSC drugs or MSC treatment technology derived from umbilical cord has been approved for marketing. Compared with bone marrow or adipose tissue, umbilical cord-derived mesenchymal stem cells have lower immunogenicity, can be used for allogeneic therapy, are easier to obtain, and are more suitable for large-scale production. Meanwhile, in the prior art a culture system containing fetal bovine serum (FBS) is usually used, posing a safety risk due to the exogenous serum introduced. Moreover, in the prior art MSC product is usually in the fresh preparation form after thaw and culture, which is difficult to maintain the cell biological activity for a long time and needs to be used or injected in time. These are the urgent issues that need to be addressed in the MSC industry at present.

### SUMMARY OF THE INVENTION

The objective of the present invention is to provide a preparation method and use of human umbilical cord-derived mesenchymal stem cells (hUC-MSCs) and cryopreservation preparation thereof.

In order to achieve the above objective, the present invention firstly provides a method for preparing hUC-MSCs.

The method for preparing hUC-MSCs provided by the present invention comprises the following steps:
(1) culturing an isolated umbilical cord tissue block, and obtaining P0 (Passage 0) mesenchymal stem cells when cell confluence reaches 40%-60% after the mesenchymal stem cells migrating from the umbilical cord tissue block;
(2) after the above step (1), subculturing the P0 mesenchymal stem cells until the cell confluence is 60%-80% to obtain P1 (Passage 1) mesenchymal stem cells;
(3) after step (2), subculturing the P1 mesenchymal stem cells until the cell confluence is 60%-80% to obtain P2 (Passage 2) mesenchymal stem cells;
(4) after step (3), performing cell cryopreservation on the P2 mesenchymal stem cells to obtain cryopreserved P2 seed bank cells;
(5) after step (4), thawing the cryopreserved P2 seed bank cells and performing subculturing until the cell confluence is 60%-80% to obtain P3 (Passage 3) mesenchymal stem cells;
(6) after step (5), subculturing the P3 mesenchymal stem cells until the cell confluence is 60%-80% to obtain P4 (Passage 4) mesenchymal stem cells;
(7) after step (6), performing cell cryopreservation on the P4 mesenchymal stem cells to obtain cryopreserved P4 working cell bank cells;
(8) after step (7), thawing the cryopreserved P4 working bank cells and performing subculturing until the cell confluence is 60-80% to obtain P5 (Passage 5) mesenchymal stem cells; and
(9) after step (8), the P5 cells are sequentially digested, washed and resuspended to obtain the hUC-MSCs.

In the above method for preparing hUC-MSCs, prior to step (1), the following steps are further included: taking the isolated umbilical cord for sterilization to obtain the sterilized umbilical cord; then cleaning the sterilized umbilical cord and cutting it into 1-2 cm small pieces to obtain small pieces of umbilical cord; then the small piece of umbilical cord is cleaned and cut into 1-2 mm³ tissue blocks.

In step (1), the isolated umbilical cord tissue blocks are cultured in the fetal bovine serum complete medium. The method for culture may specifically include the following steps: adding fetal bovine serum complete medium to the cell culture flask containing the umbilical cord tissue blocks, culturing for 4 hours upside down under the conditions of 5% CO₂ and 37° C.; continuously culturing for 24 hours with the cell culture flask upright under the conditions of 5% CO₂, saturated humidity and 37° C.; supplementing fetal bovine serum complete medium to the cell culture flask, and continuously culturing under the conditions of 5% CO₂, saturated humidity and 37° C.; after attachment-block culture of the umbilical cord tissue blocks for 7 days, sucking up and discarding the culture solution, and adding the fetal bovine serum complete medium to the cell culture flask, and continue to culture under the conditions of 5% CO₂, saturated humidity, and 37° C.

In step (2), the fetal bovine serum complete medium is used for subculturing. The method for subculturing (P0 to P1) may specifically include the following steps: sucking up and discarding all the medium, adding sodium chloride injection (0.9%) to the cell culture flask to wash once, then adding TrypLE for digestion, and after cells are completely suspended, adding sodium chloride injection (0.9%) to stop the digestion, and transferring the cell suspension to a centrifuge tube; washing each cell culture flask with sodium chloride injection (0.9%), and transferring the washed cell suspension together into a centrifuge tube; centrifuging the centrifuge tube (300 g for 8 minutes), sucking up and discarding the supernatant, resuspending the cells with fetal bovine serum complete medium equilibrated to room temperature, mixing well and taking the cell suspension for counting, calculating the total number of harvested P0 cells; seeding in cell culture flasks at a density of 18,000-20,000 cells/cm², supplementing fetal bovine serum complete medium to the standard volume of each flask, marking and placing it under the conditions of 5% CO₂, saturated humidity, and 37° C. to continue culturing.

In step (3), the fetal bovine serum complete medium is used for subculturing. The method for subculturing (P1 to P2) may specifically include the following steps: sucking up and discarding all the medium, adding sodium chloride injection (0.9%) to the cell culture flask to wash once, then adding TrypLE for digestion, and after cells are completely suspended, adding sodium chloride injection (0.9%) to stop the digestion, and transferring the cell suspension to a centrifuge tube; washing each cell culture flask with sodium chloride injection (0.9%), and transferring the washed cell suspension together into a centrifuge tube; centrifuging the centrifuge tube (300 g for 8 minutes), sucking up and discarding the supernatant, resuspending the cells with fetal bovine serum complete medium equilibrated to room temperature, mixing well and taking the cell suspension for counting, calculating the total number of harvested P1 cells; seeding in cell culture flasks at a density of 18,000-20,000 cells/cm², supplementing fetal bovine serum complete medium to the standard volume for each flask, marking and placing it under the conditions of 5% CO₂, saturated humidity, and 37° C. to continue culturing.

In step (4), the cell cryopreservation method may specifically include the following steps: sucking up and discarding all the culture solution, adding sodium chloride injection (0.9%) to the cell culture flask to wash once, then adding TrypLE for digestion, and after cells are completely suspended, adding sodium chloride injection (0.9%) to stop the digestion, and transferring the cell suspension to a centrifuge tube; washing each culture flask with sodium chloride injection (0.9%), and transferring the washed cell suspension together into a centrifuge tube; centrifuging the centrifuge tube (300 g for 8 minutes), sucking up and discarding the supernatant, resuspending the cells with cryopreservation solution A, mixing well and taking the cell suspension for counting, calculating the total number of harvested P2 cells; supplementing the required volume of cryopreservation solution A according to the counting results to make cell suspension, so that the cell concentration is 3E6 cells/mL, and adding the cell suspension to the cryopreservation tube, placing in a gradient cooling box (pre-cooled at 4° C.) and freezing in a −80° C. low-temperature refrigerator, and transferring to a liquid nitrogen storage tank within 1 month. Further, the cryopreservation solution A consists of fetal bovine serum and dimethyl sulfoxide. Furthermore, the volume ratio of the fetal bovine serum and the dimethyl sulfoxide can be 9:1.

In step (5), the thawing method may specifically include the following steps: putting the cryopreservation tube containing the P2 seed bank cells into a 37° C. water bath until the cryopreserved cells are completely thawed; then transferring the cell suspension in the cryopreservation tube to a centrifuge tube filled with serum-free complete medium, mixing well and centrifuging (300 g for 5 minutes), sucking up and discarding the supernatant, and resuspending the cells with serum-free complete medium equilibrated to room temperature. Serum-free complete medium is used for subculturing. The method of subculturing (P2 to P3) may specifically include the following steps: seeding in cell culture flasks at a density of 8,000-9,000 cells/cm², supplementing serum-free complete medium to the standard volume for each flask, marking and placing it under the conditions of 5% CO₂, saturated humidity, and 37° C. to continue culturing.

In step (6), the serum-free complete medium is used for subculturing. The method of subculturing (P3 to P4) may specifically include the following steps: sucking up and discarding all the medium, adding sodium chloride injection (0.9%) to the cell culture flask to wash once, then adding TrypLE for digestion, and after cells are completely suspended, adding sodium chloride injection (0.9%) to stop the digestion, and transferring the cell suspension to a centrifuge tube; washing each cell culture flask with sodium chloride injection (0.9%), and transferring the washed cell suspension together into a centrifuge tube; centrifuging the centrifuge tube (300 g for 8 minutes), sucking up and discarding the supernatant, resuspending the cells with serum-free complete medium equilibrated to room temperature, mixing well and taking the cell suspension for counting, calculating the total number of harvested P3 cells; seeding in cell culture flasks at a density of 6,000-8,000 cells/cm², supplementing serum-free complete medium to the standard volume for each flask, marking and placing it under the conditions of 5% CO₂, saturated humidity, and 37° C. to continue culturing.

In step (7), the cell cryopreservation method may specifically include the following steps: sucking up and discarding all the culture solution, adding sodium chloride injection (0.9%) to the cell culture flask to wash once, then adding TrypLE for digestion, and after cells are completely suspended, adding sodium chloride injection (0.9%) to stop the digestion, and transferring the cell suspension to a centrifuge tube; washing each culture flask with sodium chloride injection (0.9%), and transferring the washed cell suspension together into a centrifuge tube; centrifuging the centrifuge tube (300 g for 8 minutes), sucking up and discarding the supernatant, resuspending the cells with cryopreservation solution B so that the cell density is 8E6 cells/mL; then adding the cell suspension to the cryopreservation tube, placing in a gradient cooling box (pre-cooled at 4° C.) and freezing in a −80° C. low-temperature refrigerator, and transferring to a liquid nitrogen storage tank within 1 month. Further, the cryopreservation solution B consists of serum-free medium base, dimethyl sulfoxide and human albumin solution. Furthermore, the volume ratio of the serum-free medium base, dimethyl sulfoxide and human albumin solution (0.2 g/mL) can be 7:2:1.

In step (8), the thawing method may specifically include the following steps: putting the cryopreservation tube containing the P4 working bank cells into a 37° C. water bath until the cryopreserved cells are completely thawed; then transferring the cell suspension in the cryopreservation tube to a centrifuge tube filled with serum-free complete medium, mixing well and centrifuging (300 g for 5 minutes), sucking up and discarding the supernatant, and resuspending the cells with serum-free complete medium equilibrated to room temperature. Serum-free complete medium is used for subculturing. The method of subculturing (P4 to P5) may specifically include the following steps: seeding in cell culture flasks at a density of 8,000-11,000 cells/cm², supplementing serum-free complete medium to the standard volume for each flask, marking and placing it under the conditions of 5% CO₂, saturated humidity, and 37° C. to continue culturing.

The step (9) may specifically include the following steps: sucking up and discarding all the culture solution, adding sodium chloride injection (0.9%) to the cell culture flask to wash once, then adding TrypLE for digestion, and after cells are completely suspended, adding sodium chloride injection (0.9%) to stop the digestion, and transferring the cell suspension to a centrifuge tube; washing each cell culture flask with sodium chloride injection (0.9%), and transferring the washed cell suspension together into a centrifuge tube; centrifuging the centrifuge tube (300 g for 8 minutes), sucking up and discarding the supernatant, washing the cells with sodium chloride injection (0.9%) containing human serum albumin (0.1%), centrifuging (300 g for 8 minutes); repeating the centrifugation and washing steps; until the third washing, resuspending the cells with sodium chloride injection (0.9%) containing human serum albumin (0.1%) to obtain a stock solution of hUC-MSCs, which contains the hUC-MSCs.

In the above method, the fetal bovine serum complete medium consists of DMEM/F12 basal medium and fetal bovine serum. The volume ratio of the DMEM/F12 basal medium and the fetal bovine serum can be 9:1.

The serum-free complete medium consists of a serum-free medium base for mesenchymal stem cells and a serum-free medium supplement for mesenchymal stem cells. The volume ratio of the serum-free medium base for mesenchymal stem cells and the serum-free medium supplement for mesenchymal stem cells can be 100:1.

In order to achieve the above objective, the present invention further provides hUC-MSCs prepared according to the above method.

In order to achieve the above objective, the present invention further provides the use of the above hUC-MSCs in the preparation of a cryopreservation preparation of hUC-MSCs.

In order to achieve the above objective, the present invention further provides a cryopreservation preparation of hUC-MSCs.

The cryopreservation preparation of hUC-MSCs provided by the present invention includes the above hUC-MSCs and a cell cryoprotectant.

Further, the cell cryoprotectant consists of a multiple electrolyte solution, dimethyl sulfoxide, a dextran 40 sodium chloride injection and a human albumin solution (0.2 g/mL).

The concentration of the hUC-MSCs in the cryopreservation preparation can be 2.5×10⁶-1×10⁷ cells/mL, specifically can be 2.5×10⁶ cells/mL, 5×10⁶ cells/mL and 1×10⁷ cells/mL.

Furthermore, the volume ratio of the multiple electrolyte solution, the dimethyl sulfoxide, the dextran 40 sodium chloride injection and the human albumin solution (0.2 g/mL) can be (70-80):(5-10):(5-10):(5-10) or 70:(5-10):(5-10):(5-10) or 80:(5-10):(5-10):(5-10) or (70-80):5:(5-10):(5-10) or (70-80):10:(5-10):(5-10) or (70-80):(5-10):5:(5-10) or (70-80):(5-10):10:(5-10) or (70-80):(5-10):(5-10):5 or (70-80):(5-10):(5-10):10.

In a specific embodiment of the present invention, the volume ratio of the multiple electrolyte solution, the dimethyl sulfoxide, the dextran 40 sodium chloride injection and the human albumin solution (0.2 g/mL) is 80:5:10:5 or 70:10:10:10 or 80:5:5:10 or 80:10:5:5.

In order to achieve the above objective, the present invention further provides a method for preparing the above cryopreservation preparation of hUC-MSCs.

The method for preparing the cryopreservation preparation of hUC-MSCs provided by the present invention comprises the following steps: resuspending the above hUC-MSCs with a cell cryoprotectant, and then cooling the obtained cell suspension to obtain the cryopreservation preparation of hUC-MSCs.

Further, the procedure of the cooling treatment is as follows:
Step 1: initial temperature to 4° C.;
Step 2: 1° C./min down to −5° C.;
Step 3: 20° C./min until the cavity temperature is −40° C.;
Step 4: 10° C./min until the cavity temperature is −20° C.;
Step 5: 2° C./min until the sample drops to −40° C.;
Step 6: 10° C./min until the sample drops to −80° C.; and
Step 7: end.

In order to achieve the above objective, the present invention further provides a new use of the above hUC-MSCs or the above cryopreservation preparation of hUC-MSCs or the cryopreservation preparation of hUC-MSCs prepared according to the above method.

The present invention provides the use of the hUC-MSCs or the cryopreservation preparation of hUC-MSCs or the cryopreservation preparation of hUC-MSCs prepared according to the above method in any of the following N1)-N10):
N1) preparation of products for the prevention and/or treatment of diseases caused by novel coronaviruses;
N2) preparation of products for the prevention and/or treatment of idiopathic pulmonary interstitial fibrosis;
N3) preparation of products for the prevention and/or treatment of acute lung injury;
N4) preparation of products for the prevention and/or treatment of liver fibrosis;
N5) preparation of products for the prevention and/or treatment of Crohn's disease;
N6) prevention and/or treatment of diseases caused by novel coronaviruses;
N7) prevention and/or treatment of idiopathic pulmonary interstitial fibrosis;
N8) prevention and/or treatment of acute lung injury;
N9) prevention and/or treatment of liver fibrosis; and
N10) prevention and/or treatment of Crohn's disease.

In order to achieve the above objective, the present invention further provides a product, the active ingredient of which is the hUC-MSCs or the above cryopreservation preparation of hUC-MSCs, or the cryopreservation preparation of hUC-MScs prepared according to the above method; the function of the product is any one of M1)-M5):
M1) prevention and/or treatment of diseases caused by novel coronavirus;
M2) prevention and/or treatment of idiopathic pulmonary interstitial fibrosis;
M3) prevention and/or treatment of acute lung injury;
M4) prevention and/or treatment of liver fibrosis; and
M5) prevention and/or treatment of Crohn's disease.

In order to achieve the above objective, the present invention finally provides a method for treating a disease caused by a novel coronaviruses or idiopathic pulmonary interstitial fibrosis or acute lung injury or liver fibrosis or Crohn's disease.

The method for treating a disease caused by a novel coronaviruses or idiopathic pulmonary interstitial fibrosis or acute lung injury or liver fibrosis or Crohn's disease, comprises the following steps: administering the above hUC-MSCs or the above cryopreservation preparation of hUC-MSCs or the above cryopreservation preparation of hUC-MSCs to a patient with a disease caused by a novel coronavirus or with idiopathic pulmonary interstitial fibrosis or with acute lung injury or with liver fibrosis or with Crohn's disease to allow the patient to be treated.

In any of the above Uses or products, the prevention and/or treatment of diseases caused by novel coronaviruses is embodied in the reduction of viral load in the lungs and/or improvement of the inflammatory infiltration around small blood vessels and/or improvement of the inflammatory infiltration around bronchioles and/or improvement of lung bronchiole epithelial cell degeneration.

The prevention and/or treatment of idiopathic pulmonary interstitial fibrosis is embodied in the improvement of the lung function damage caused by fibrosis and/or inhibition of the expression of pro-fibrosis factors (such as HYP, MMP-2, TIMP-1) and/or improvement of collagen deposition in lung tissue and/or improvement of pulmonary interstitial or tracheal or perivascular or alveolar inflammatory infiltration and fibrosis.

The prevention and/or treatment of acute lung injury is embodied in the reduction of lung water content and/or improvement of pulmonary edema and/or improvement of acute respiratory distress syndrome (ARDS) and/or improvement of lung inflammation and/or improvement of lung tissue inflammatory infiltration and/or improvement of the lesion degree of lymphoid tissue hyperplasia.

The prevention and/or treatment of liver fibrosis is embodied in the improvement of the four indexes of liver fibrosis and/or improvement of liver structure and/or improvement of liver collagen deposition.

The prevention and/or treatment of Crohn's disease is embodied in a reduction in the area of colonic ulcers and/or reduction in the incidence of ulcers and/or alleviation of colonic injury.

In any of the above Uses or products, the product can be a drug.

In any of the above Uses or product methods, the disease caused by the novel coronavirus is novel coronavirus pneumonia (COVID-19). The novel coronavirus is SARS-COV-2, specifically the SARS-COV-2 (HRB26) strain.

### BRIEF DESCRIPTION OF THE FIGURE

FIG. 1 is a morphological diagram of hUC-MSCs.

FIG. 2 is a staining diagram of the differentiation of hUC-MSCs. From top to bottom, after 21 days of osteogenic induction and differentiation, Alizarin Red-S staining is positive; after 14 days of adipogenic induction and differentiation, Oil Red-O staining is positive; after 21 days of chondrogenic tissue induction and differentiation, Alcian blue staining is positive after paraffin section.

FIG. 3 shows the viability of cells after thawing.

FIG. 4 is a graph showing the expression level of IDO gene mRNA in hUC-MSCs.

FIG. 5 is a graph showing the expression level of CCL2 gene mRNA in hUC-MSCs.

FIG. 6 is a graph showing the expression level of CXCL10 gene mRNA in hUC-MSCs.

FIGS. 7A-7E shows the effect of the test product on the pathological changes of the lung tissue of the BLM-induced pulmonary fibrosis model rat. Note: HE staining, 100×. 7A. in the normal control group, no obvious lesions are seen under the microscope in the right upper lobe of the lung; 7B. in the model control group, alveolar inflammatory cell infiltration (obvious) and interstitial/alveolar fibrosis (obvious) are seen in the right upper lobe of the lung; 7C. in the C1 low-dose group, alveolar inflammatory cell infiltration (moderate) and interstitial/alveolar fibrosis (moderate) are seen in the right upper lobe of the lung; 7D. in the C1 medium-dose group, alveolar inflammatory cell infiltration (mild) and interstitial/alveolar fibrosis (moderate) are seen in the right upper lobe of the lung; 7E. in the C1 high-dose group, alveolar inflammatory cell infiltration (mild) and interstitial/alveolar fibrosis (mild) are seen under the microscope in the right upper lobe of the lung.

FIGS. 8A-8E shows the effect of the test product on the lung histopathology of ALI model rats. Note: HE staining, 40×. 8A. in the normal control group, no obvious abnormal changes are seen in the right lung. 8B. in the model control group, interstitial/alveolar inflammatory cell infiltration and broncho-associated lymphoid tissue hyperplasia in the right lung. 8C. in the C1 low-dose group, interstitial/alveolar inflammatory cell infiltration and broncho-associated lymphoid tissue hyperplasia in the right lung. 8D. In the C1 medium-dose group, interstitial/alveolar inflammatory cell infiltration and broncho-associated lymphoid tissue hyperplasia in the right lung. 8E. in the C1 high-dose group, interstitial/alveolar inflammatory cell infiltration and broncho-associated lymphoid tissue hyperplasia in the right lung.

### BRIEF DESCRIPTION OF THE FIGURE

The present invention will be further described in detail below in conjunction with specific embodiments, and the given examples are only for clarifying the present invention, not for limiting the scope of the present invention. The examples provided below can be used as a guide for those skilled in the art to make further improvements, and are not intended to limit the present invention in any way.

The experimental methods in the following examples, unless otherwise specified, are conventional methods, and are carried out according to the techniques or conditions described in the literature in this field or according to the product instructions. The materials and reagents used in the following examples can be obtained from commercial sources unless otherwise specified.

Fetal bovine serum complete medium formula: 10% fetal bovine serum (Biological Industries (BI), catalog number: 04-001-1ACS)+90% DMEM/F12 basal medium (Biological Industries (BI), catalog number: 04-172-1ACS).

Serum-free complete medium formula: 5 mL mesenchymal stem cell serum-free medium supplement (Youkang Biotechnology, catalog number: NC0103.S) is added to each 500 mL mesenchymal stem cell serum-free medium base (Youkang Biotechnology, catalog number: NC0103).

Chondrogenesis Induced Differentiation Complete Medium: Biological Industries (BI), catalog number: 05-220-1B.

Osteogenic Induction Medium: Biological Industries (BI), catalog number: 05-440-1B.

Adipogenic Induction Medium: Biological Industries (BI), catalog number: 05-330-1B.

TrypLE: Thermo Fisher (GIBCO), catalog number: 12563-029.

Mouse IgG1-FITC: BD Bioscience, catalog number: 555748.

antiCD19-FITC: BD Bioscience, catalog number: 555412.

antiCD34-FITC: BD Bioscience, catalog number: 555821.

Mouse IgG1-PE: BD Bioscience, catalog number: 554680.

antiCD11b-PE: BD Bioscience, catalog number: 555388.

antiCD73-PE: BD Bioscience, catalog number: 550257.

antiCD90-PE: BD Bioscience, catalog number: 555596.

antiCD45-PE: BD Bioscience, catalog number: 555483.

antiCD105-PE: BD Bioscience, catalog number: 560839.

antiHLA-DR-PE: BD Bioscience, catalog number: 555812.

Multiple electrolyte injection: Shanghai Baite Medical Supplies Co., Ltd., catalog number: National Drug Approval H20000475.

Dimethyl sulfoxide (DMSO): Hunan Jiudian Pharmaceutical Co., Ltd., catalog number: Xiangshiyaofuzhunzi F20090010.

Dextran 40 Sodium Chloride Injection: Shijiazhuang No. 4 Pharmaceutical Co., Ltd., catalog number: National Drug Approval H13022493.

Human albumin solution (0.2 g/mL): Switzerland Jet Behring Biological Products Co., Ltd.

IFN-γ: Inshore Organisms, catalog number: C014.

0.9% Sodium Chloride Injection: Shijiazhuang No. 4 Biological Co., Ltd., catalog number: National Drug Approval H13023201.

### Example 1. Preparation and Identification of hUC-MSCs

(1) Preparation of hUC-MSCs
I. Isolation of hUC-MSCs

1. The isolated umbilical cords of full-term newborns without congenital diseases (the newborns had no infectious diseases such as hepatitis, syphilis, and AIDS, and the mothers and their families had informed consent for the use of umbilical cords in experimental research) were put into the beaker filled with 50 mL of 75% ethanol and sterilized for 10 s. In a beaker with 50 mL of 75% ethanol, sterilize for 10 s. Then rinsed with 30 mL 0.9% Sodium Chloride Injection (Shijiazhuang No. 4 Pharmaceutical Co., Ltd., National Drug Approval H13023201) three times.

2. After completing step 1, 50 mL of 0.9% sodium chloride injection was added into the kidney-shaped dish, and then the umbilical cord was put into the kidney-shaped dish to remove surface blood stains, and then the umbilical cord was cut into 1-2 cm pieces with surgical scissors. The blood was further drained, and the waste liquid was discarded.

3. After completing step 2, the small section of umbilical cord was put into a beaker filled with 30 mL 0.9% sodium chloride injection, washed three times repeatedly, and then under clean conditions, the small section of umbilical cord after cleaning was placed in a dry glass beaker, and the umbilical cord was cut into pieces of about 1-2 mm³ with surgical scissors, and uniformly inoculated in T75 cell culture flasks. 4. After completing step 3, 4 mL of fetal bovine serum complete medium was added into the T75 cell culture flask, and placed upside down in a 5% CO₂, 37° C. incubator. After 4 hours, the culture flask was placed upright in 5% CO₂, saturated humidity, and continue to culture in a 37° C. incubator. After 24 hours, each T75 culture flask was supplemented with 11 mL of fetal bovine serum complete medium, placed in 5% CO₂, saturated humidity, and continued to culture in a 37° C. incubator. After the umbilical cord tissue blocks were attachment-block cultured for 7 days, the culture solution was sucked up and discarded, and 15 mL of fetal bovine serum complete medium was added to each T75 culture flask, placed in 5% CO₂, saturated humidity, and continued to culture in a 37° C. incubator. During culture, mesenchymal stem cells migrated out from the umbilical cord tissue blocks.

### II. Seed Bank Establishment (P0-P2)

1. When the cell confluence in the whole flask reached 40%-60% after 9-13 days of attachment-block culture of umbilical cord tissue blocks, the P0 mesenchymal stem cells were obtained, and then the P0-P1 subculturing was carried out. The specific method was as follows: all the medium was sucked up and discarded, and 5 mL 0.9% sodium chloride injection was added to each T75 culture flask to wash once, then 2.5 mL TrypLE was added, digested for about 2 minutes, and 5 mL 0.9% sodium chloride injection was added after the cells were completely suspended to terminate the digestion, and the cell suspension was transferred to a centrifuge tube. Each culture flask was washed with 5 mL 0.9% sodium chloride injection, and the washed cell suspension together was transferred into a centrifuge tube. The centrifuge tube was centrifuged at 300 g for 8 minutes, the supernatant was sucked up and discarded after centrifugation, the cells was resuspended with fetal bovine serum complete medium equilibrated to room temperature, after mixing well, the cell suspension was taken for counting, and the total number of harvested P0 cells was calculated. Seeded in culture flasks at a density of 18,000-20,000 cells/cm². Each flask was supplemented with fetal bovine serum complete medium to the standard volume (T75: 15 mL; T175: 35 mL), marked and placed in a 5% CO₂, saturated humidity, 37° C. incubator to continue culturing.

2. After step 1 was completed, when the cell confluence in the whole flask reached 60%-80% after 48-72 hours of inoculation, the P1 mesenchymal stem cells were obtained, and then the P1-P2 subculturing was carried out. The specific method was as follows: all the culture solution was sucked up and discarded, and 0.9% sodium chloride injection (T75: 5 mL; T175: 10 mL) was added to each flask to wash once, then TrypLE (T75: 2.5 mL; T175: 5 mL) was added, digested for about 2 minutes, and 0.9% sodium chloride injection (T75: 5 mL; T175: 10 mL) was added after the cells were completely suspended to terminate the digestion, and the cell suspension was transferred to a 250 mL centrifuge tube. Each culture flask was washed with 0.9% sodium chloride injection (T75: 5 mL; T175: 10 mL), and the washed cell suspension together was transferred into a centrifuge tube. The centrifuge tube was centrifuged at 300 g for 8 minutes, the supernatant was sucked up and discarded after centrifugation, the cells was resuspended with fetal bovine serum complete medium equilibrated to room temperature, after mixing well, the cell suspension was taken for counting, and the total number of harvested P1 cells was calculated. Seeded in culture flasks at a density of 18,000-20,000 cells/cm². Each flask was supplemented with fetal bovine serum complete medium to the standard volume (T175: 35 mL; T525: 105 mL), marked and placed in a 5% CO₂, saturated humidity, 37° C. incubator to continue culturing.

3. After step 2 was completed, when the cell confluence in the whole flask reached 60%-80% after 48-72 hours of inoculation, the P2 mesenchymal stem cells were obtained, and then cryopreservation of P2 seed bank cells was carried out. The specific method was as follows: all the culture solution was sucked up and discarded, and 0.9% sodium chloride injection (T175: 10 mL; T525: 30 mL) was added to each flask to wash once, then TrypLE (T175: 5 mL; T525: 15 mL) was added, digested for about 2 minutes, and 0.9% sodium chloride injection (T175: 10 mL; T525: 30 mL) was added after the cells were completely suspended to terminate the digestion, and the cell suspension was transferred to a 250 mL centrifuge tube. Each culture flask was washed with 0.9% sodium chloride injection (T175: 10 mL; T525: 30 mL), and the washed cell suspension together was transferred into a centrifuge tube. The centrifuge tube was centrifuged at 300 g for 8 minutes, the supernatant was sucked up and discarded after centrifugation, and the cells were resuspended in the cryopreservation solution (fetal bovine serum: DMSO=9:1 (volume ratio)). After mixing well, the cell suspension was taken for counting, and the total number of harvested P2 cells was calculated. According to the counting results, the required volume of cryopreservation solution was supplemented to make a cell suspension, and the cell concentration was adjusted to about 3E6 cells/mL, and the cell suspension was added to the cryopreservation tube, 1.5 mL/tube. It was placed in a pre-cooled gradient cooling box at 4° C., placed in a −80° C. low-temperature refrigerator, and frozen. It was transferred to a liquid nitrogen storage tank within 1 month.

### III. Working Bank Establishment (P2-P4)

1. The cryopreserved P2 cells were taken for thawing. The specific method was as follows: the cryopreservation tube containing the P2 cells was put into a 37° C. water bath, and the thawing situation was randomly checked in 1-3 minutes until the cryopreserved cells were completely thawed.

2. After completing step 1, the cell suspension in the cryopreservation tube was transferred to a centrifuge tube filled with serum-free complete medium, after mixing well and centrifuged at 300 g for 5 minutes, the cells were resuspended with serum-free complete medium equilibrated to room temperature.

3. After completing step 2, P2-P3 subculturing was carried out. The specific method was as follows: the cells were seeded in cell culture flasks at a density of 8,000-9,000 cells/cm², and serum-free complete medium was supplemented to each flask to a standard volume (T175: 35 mL; T525: 105 mL), marked and placed in 5% CO₂, saturated humidity, 37° C. incubator to continue culturing. 4. After step 3 was completed, when the cell confluence reached 60%-80% after 48-72 hours of inoculation, the P3 mesenchymal stem cells were obtained, and then the P3-P4 subculturing was carried out. The specific method was as follows: all the culture solution was sucked up and discarded, and 0.9% sodium chloride injection (T175: 10 mL; T525: 30 mL) was added to each flask to wash once, then TrypLE (T175: 5 mL; T525: 15 mL) was added, digested for about 2 minutes, and 0.9% sodium chloride injection (T175: 10 mL; T525: 30 mL) was added after the cells were completely suspended to terminate the digestion, and the cell suspension was transferred to a 250 mL centrifuge tube. Each culture flask was washed with 0.9% sodium chloride injection (T175: 10 mL; T525: 30 mL), and the washed cell suspension together was transferred into a centrifuge tube. The centrifuge tube was centrifuged at 300 g for 8 minutes, the supernatant was sucked up and discarded after centrifugation, the cells was resuspended with serum-free complete medium equilibrated to room temperature, after mixing well, the cell suspension was taken for counting, and the total number of harvested P3 cells was calculated. Seeded in culture flasks at a density of 6,000-8,000 cells/cm². Each flask was supplemented with serum-free complete medium to the standard volume (T525: 105 mL; T875: 175 mL), marked and placed in a 5% CO₂, saturated humidity, 37° C. incubator to continue culturing.

5. After step 4 was completed, when the cell confluence reached 60%-80% after 48-72 hours of inoculation, the P4 mesenchymal stem cells were obtained, and then cryopreservation of P4 working bank cells was carried out. The specific method was as follows: all the culture solution was sucked up and discarded, and 0.9% sodium chloride injection (T175: 10 mL; T525: 30 mL) was added to each flask to wash once, then TrypLE (T175: 5 mL; T525: 15 mL) was added, digested for about 2 minutes, and 0.9% sodium chloride injection (T175: 10 mL; T525: 30 mL) was added after the cells were completely suspended to terminate the digestion, and the cell suspension was transferred to a 250 mL centrifuge tube. Each culture flask was washed with 0.9% sodium chloride injection (T175: 10 mL; T525: 30 mL), and the washed cell suspension together was transferred into a centrifuge tube. The centrifuge tube was centrifuged at 300 g for 8 minutes, the supernatant was sucked up and discarded after centrifugation, and the cells were resuspended in the cryopreservation solution (serum-free medium base: DMSO: human albumin solution (0.2 g/mL)=7:2:1 (volume ratio)). After mixing well, the cell suspension was taken for counting, and the total number of harvested P4 cells was calculated. According to the counting results, the required volume of cryopreservation solution was supplemented to make a cell suspension, and the cell concentration was adjusted to about 3E6 cells/mL, and the cell suspension was added to the cryopreservation tube, 1.5 mL/tube. It was placed in a pre-cooled gradient cooling box at 4ºC, placed in a −80° C. low-temperature refrigerator, and frozen. It was transferred to a liquid nitrogen storage tank within 1 month.

### IV. Preparation of Cell Stock Solution (P4-P5)

1. The cryopreserved P4 cells were taken for thawing. The specific method was as follows: the cryopreservation tube containing the P4 cells was put into a 37° C. water bath, and the thawing situation was randomly checked in 1-3 minutes until the cryopreserved cells were completely thawed.

2. After completing step 1, the cell suspension in the cryopreservation tube was transferred to a centrifuge tube filled with serum-free complete medium, after mixing well and centrifuged at 300 g for 5 minutes, the cells were resuspended with serum-free complete medium equilibrated to room temperature.

3. After completing step 2, P4-P5 subculturing was carried out. The specific method was as follows: the cells were seeded in cell culture flasks at a density of 8,000-11,000 cells/cm², and serum-free complete medium was supplemented to each flask to a standard volume (T525: 105 mL), marked and placed in 5% CO₂, saturated humidity, 37° C. incubator to continue culturing.

4. After step 3 was completed, when the cell confluence reached 60%-80% after 48-72 hours of inoculation (the cell morphology was shown in FIG. 1), the P5 mesenchymal stem cells were obtained, and then the stock solution was prepared. The specific method was as follows: the culture solution was sucked up and discarded from all the culture flasks, and 0.9% sodium chloride injection (T525: 30 mL) was added to each flask to wash once, then TrypLE (T525: 15 mL) was added, digested for about 2 minutes, and 0.9% sodium chloride injection (T525: 30 mL) was added after the cells were completely suspended to terminate the digestion, and the cell suspension was transferred to a 250 mL centrifuge tube. Each culture flask was washed with 0.9% sodium chloride injection (T525: 30 mL), and the washed cell suspension together was transferred into a centrifuge tube, centrifuged at 300 g for 8 minutes, and the supernatant was sucked up and discarded after centrifugation. The cells were washed with 0.9% sodium chloride injection containing 0.1% human serum albumin, and centrifuged at 300 g for 8 minutes. The centrifugation wash step was repeated. At the third washing, the cells were resuspended with 0.9% sodium chloride injection containing 0.1% human serum albumin to obtain the stock solution of hUC-MSCs.

According to the above method, three umbilical cords from different sources (respectively numbered Y200001, Y200003, and Y210001) were used as raw materials to prepare stem cell stock solutions. The umbilical cords numbered Y200001 and Y200003 were obtained from Hubei Provincial People's Hospital, and the umbilical cord numbered Y210001 was obtained from Tongji Hospital, Tongji Medical College, Huazhong University of Science & Technology.

(2) Identification of hUC-MSCs in the Stock Solution
I. Differentiation Identification of hUC-MSCs in Stock Solution

1. Stem cell stock solution from three different umbilical cord-deriveds (respectively numbered Y200001, Y200003, and Y210001) were taken as examples. 1 mL of cells with a concentration of 1-2E6/mL was added into a 15 mL centrifuge tube, and centrifuged at 300 g for 5 min. Then 1 mL of chondrogenesis induced differentiation complete medium was added to resuspend the cells, centrifuged at 200 g for 5 min at room temperature, placed them in a 37° C., 5% CO₂ incubator for culture, and replaced with chondrogenesis induced differentiation complete medium every 3 days. After continuous induction for 21 days, the chondrocytes were fixed in 4% paraformaldehyde, embedded in paraffin and sectioned, and finally stained with Alcian blue.

2. After step 1 was completed, cells were seeded in a well plate at a density of 8000 cells/mL, cultured in a 37° C., 5% CO₂ incubator, and divided into the following two groups for treatment:

Osteogenic induction group: when the cell fusion rate reached 80%, the medium was sucked up and discarded, the osteogenic induction medium was added, fresh osteogenic induction medium was changed every 3 days, cultured for 21 days, and stained with Alizarin Red-S.

Adipogenic induction group: when the cell fusion rate reached 100%, the medium was sucked up and discarded, adipogenic induction medium was added, and fresh adipogenic induction medium was replaced every 2 days. Cultured for 14 days and stained with Oil Red O.

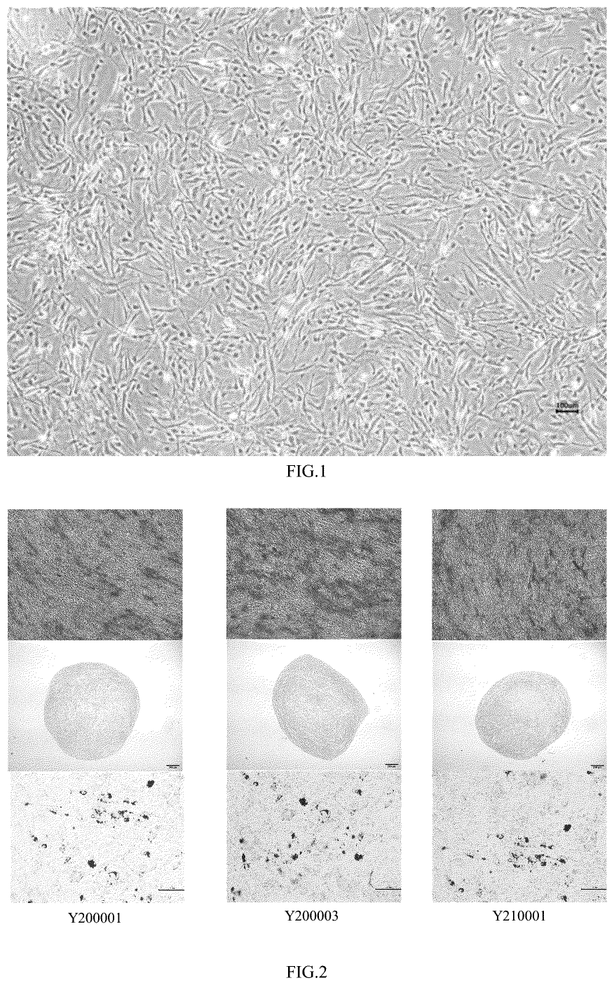

The result was shown in FIG. 2. The results showed that: the hUC-MSCs prepared by the method of the present invention had good differentiation performance and met the characteristics of stem cells.

II. Identification of Surface Markers of hUC-MSCs in the Stock Solution

Stem cell stock solution from three different umbilical cord-deriveds (respectively numbered Y200001, Y200003, and Y210001) were taken as examples, and the cell concentration was adjusted to 2×10⁶/mL respectively. The flow tubes were taken and Mouse IgG1-FITC, antiCD19-FITC, antiCD34-FITC, Mouse IgG1-PE, antiCD11b-PE, antiCD73-PE, antiCD90-PE, antiCD45-PE, antiCD105-PE, and antiHLA-DR-PE were added in sequence, respectively. The amount of antibody added was 5 μL, and 100 μL of the cell suspension to be tested was added respectively, shaken and mixed, and incubated at room temperature in the dark for 20 min. 2 mL 1×PBS was added to each tube for washing, and centrifuged at 1200 rpm for 5 min. The supernatant was discarded, 200 μL 1×PBS was added to resuspend, mixed well, and BD FACSCalibur flow cytometer was used to detect 10,000 cells per sample.

The results were shown in Table 1. The results showed that the surface expression of CD73, CD90 and CD105 on the surface of the mesenchymal stem cells prepared by the method of the present invention were all greater than 95% and positive, while CD34, CD45, CD11b, CD19 and HLA-DR were all less than 2% and negative. There was no change in the cell surface markers after cryopreservation, in line with the characteristics of stem cell surface markers.

TABLE 1
Detection of surface markers of mesenchymal stem cells
in stock solution

| umbilical | | | |
|---|---|---|---|
| cord-derived | Y200001 | Y200003 | Y210001 |
| CD19 | 0.69% | 0.26% | 0.79% |
| CD34 | 0.34% | 0.11% | 0.79% |
| CD11b | 0.15% | 0.19% | 0.84% |
| CD73 | 99.81% | 99.95% | 99.07% |
| CD90 | 99.81% | 99.81% | 99.99% |
| CD45 | 0.07% | 0.23% | 0.88% |
| CD105 | 98.45% | 98.81% | 99.12% |
| HLA-DR | 0.10% | 0.14% | 0.35% |

### Example 2. Preparation and Performance Testing of Cryopreservation Preparation of hUC-MSCs

I. Preparation of a Cryopreservation Preparation of hUC-MSCs

1. The stock solution of mesenchymal stem cells prepared in Example 1 was centrifuged at 300 g for 8 minutes, all the supernatant was sucked up and discarded after centrifugation, and the cell pellet was collected. Then the cell pellet was resuspended with the cell cryoprotectant (cryogenic solution) and cell density grouping in Table 2, respectively.

TABLE 2
Cell cryoprotectant and cell density

| Cryo- | | | |
|---|---|---|---|
| preservation | | | |
| solution | | Density | |
| group | | group | Cell |
| number | Freezing liquid formula | number | density |
| C1 | 80% (volume fraction) | G1 | 1E7/mL |
| | multiple electrolyte injection + | | |
| | 5% (volume fraction) DMSO + | G2 | 5E6/mL |
| | 10% (volume fraction) dextran | G3 | 2.5E6/mL |
| | 40 sodium chloride injection + | | |
| | 5% (volume fraction) human | | |
| | albumin solution (0.2 g/mL) | | |
| C2 | 70% (volume fraction) | G4 | 1E7/mL |
| | multiple electrolyte injection + | G5 | 5E6/mL |
| | 10% (volume fraction) DMSO + | G6 | 2.5E6/mL |
| | 10% (volume fraction) | | |
| | dextran 40 sodium chloride | | |
| | injection + 10% (volume | | |
| | fraction) human albumin | | |
| | solution (0.2 g/mL) | | |
| C3 | 80% (volume fraction) | G7 | 1E7/mL |
| | multiple electrolyte injection + | G8 | 5E6/mL |
| | 5% (volume fraction) DMSO + | G9 | 2.5E6/mL |
| | 5% (volume fraction) dextran | | |
| | 40 sodium chloride injection + | | |
| | 10% (volume fraction) human | | |
| | albumin solution (0.2 g/mL) | | |
| C4 | 80% (volume fraction) | G10 | 1E7/mL |
| | multiple electrolyte injection + | G11 | 5E6/mL |
| | 10% (volume fraction) DMSO + | G12 | 2.5E6/mL |
| | 5% (volume fraction) dextran | | |
| | 40 sodium chloride injection + | | |
| | 5% (volume fraction) human | | |
| | albumin solution (0.2 g/mL) | | |

2. After step 1 was completed, the well-mixed cell suspension of each group was drawn into a 50 mL syringe and injected into a cryopreservation bag, each bag was filled with 12 mL. After bagging, the air in the cryopreservation bag was evacuated, and heat sealing was performed at a place 0.5 cm from the tube near the cryopreservation bag. The cryopreservation bag was put into the cryopreservation folder, and put it into the cryopreservation rack in the programmed cooling instrument (Thermo Fisher Scientific (China) Co., Ltd., model: 7453). The valve of the liquid nitrogen tank was opened, and the following cooling program on the computer was set and confirmed:
Step 1: Initial temperature to 4° C.;
Step 2: 1° C./min down to −5° C.;
Step 3: 20° C./min until the cavity temperature was −40° C.;
Step 4: 10° C./min until the cavity temperature was −20° C.;
Step 5: 2° C./min until the sample dropped to −40° C.;
Step 6: 10° C./min until the sample dropped to −80° C.;
Step 7: End.

The program was runed to cool down.

3. after completing step 2, the cryopreservation folder was transferred to a liquid nitrogen tank transfer tank for storage.

II. Detection of Cell Viability in Cryopreservation Preparation of hUC-MSCs

Stem cell solution from three different umbilical cord-deriveds (respectively numbered Y200001, Y200003, and Y210001) were taken as examples. they were prepared into 12 groups of cryopreserved preparations of mesenchymal stem cells according to the different cryopreservation solutions and densities listed in step 1 respectively, and were taken out after one week of cryopreservation and thawed at 37° C. The cells in the preparation were mixed evenly, 20 μL of the sample was taken and mixed with 20 μL of AO/PI fluorescent dye evenly, 20 μL was pipetted into the Countstar counting plate, and the fluorescence counter was used for analysis.

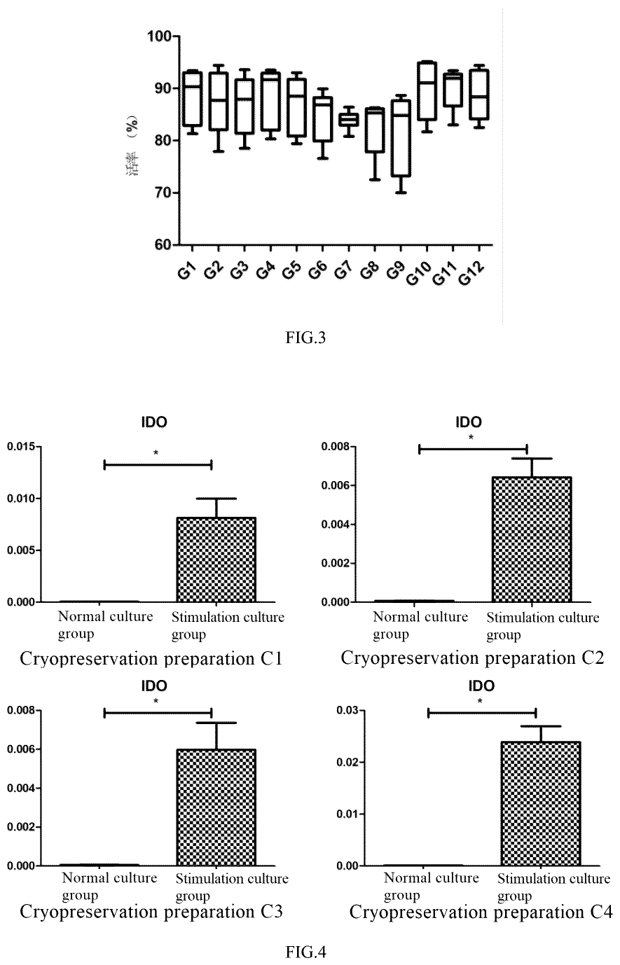

The result was shown in FIG. 3. The results showed that there was no significant difference in the activity rate of the preparations in the 12 groups, and the average values were all higher than 80%. It showed that the cell viability of the cell preparations prepared according to the cryopreservation density of 2.5×10⁶ cells/mL to 1×10⁷ cells/mL in the four kinds of cryopreservation solutions in the present invention was good after thawing.

III. Identification of Surface Markers of Different Cryopreservation Preparations of hUC-MSCs

Stem cell stock solution from three different umbilical cord-deriveds (respectively numbered Y200001, Y200003, and Y210001) were taken as examples, the cryopreserved preparations of mesenchymal stem cells were prepared according to the different cryopreservation solutions listed in step 1 at a density of 5E6/mL. After the cryopreserved cells were thawed, the cell concentration was adjusted to 2×10⁶/mL respectively. The flow tubes were taken and Mouse IgG1-FITC, antiCD19-FITC, antiCD34-FITC, Mouse IgG1-PE, antiCD11b-PE, antiCD73-PE, antiCD90-PE, antiCD45-PE, antiCD105-PE, and antiHLA-DR-PE were added in sequence, respectively. The amount of antibody added was 5 μL, and 100 μL of the cell suspension to be tested was added respectively, shaken and mixed, and incubated at room temperature in the dark for 20 min. 2 mL 1×PBS was added to each tube for washing, and centrifuged at 1200 rpm for 5 min. The supernatant was discarded, 200 μL 1×PBS was added to resuspend, mixed well, and BD FACSCalibur flow cytometer was used to detect 10,000 cells per sample.

The results were shown in Table 3-Table 5. The results showed that the surface expression of CD73, CD90 and CD105 on the stem cells of the cryopreservation preparation prepared by the method of the present invention were all greater than 95%, which was positive; CD34, CD45, CD11b, CD19 and HLA-DR were all less than 2%, which was negative. After cryopreservation, the cell surface markers did not change, which was consistent with the characteristics of stem cell surface markers.

TABLE 3
Detection of cell surface markers after cryopreservation of
cryopreserved preparations of mesenchymal stem cells prepared
from Y200001 source umbilical cord

| | Cell source | | | |
|---|---|---|---|---|
| | Y200001 | | | |
| | Cryopreservation solution | | | |
| | C1 | C2 | C3 | C4 |
| CD19 | 0.56% | 0.88% | 1.12% | 0% |
| CD34 | 0.95% | 1.30% | 1.32% | 0.02% |
| CD11b | 0.93% | 0.59% | 1.23% | 0.42% |
| CD73 | 99.52% | 99.68% | 99.52% | 99.72% |
| CD90 | 99.96% | 99.84% | 100% | 99.70% |
| CD45 | 0.83% | 1.14% | 0.87% | 0.70% |
| CD105 | 98.07% | 98.54% | 98.47% | 95.29% |
| HLA-DR | 1.37% | 0.48% | 1.05% | 1.35% |

TABLE 4
Detection of cell surface markers after cryopreservation of
cryopreserved preparations of mesenchymal stem cells
prepared from Y200003 source umbilical cord

| | Cell source | | | |
|---|---|---|---|---|
| | Y200003 | | | |
| | Cryopreservation solution | | | |
| | C1 | C2 | C3 | C4 |
| CD19 | 0.02% | 1.31% | 0.06% | 0.78% |
| CD34 | 0.13% | 0% | 0.03% | 0.07% |
| CD11b | 0% | 0.64% | 0.16% | 0.06% |
| CD73 | 96.77% | 99.80% | 99.25% | 99.86% |
| CD90 | 99.97% | 100% | 99.98% | 99.96% |
| CD45 | 0.03% | 0.13% | 0.07% | 0.14% |
| CD105 | 98.03% | 98.83% | 96.34% | 99.00% |
| HLA-DR | 0.02% | 0.41% | 0.12% | 0.04% |

TABLE 5
Detection of cell surface markers after cryopreservation of
cryopreserved preparations of mesenchymal stem cells
prepared from Y210001 source umbilical cord

| | Cell source | | | |
|---|---|---|---|---|
| | Y210001 | | | |
| | Cryopreservation solution | | | |
| | C1 | C2 | C3 | C4 |
| CD19 | 0.48% | 1.55% | 1.33% | 0.05% |
| CD34 | 0.02% | 0.06% | 1.05% | 0.05% |
| CD11b | 0.51% | 0.98% | 1.37% | 0.39% |
| CD73 | 99.57% | 99.79% | 97.97% | 99.83% |
| CD90 | 99.98% | 99.87% | 99.89% | 100% |
| CD45 | 0.16% | 0.41% | 1.65% | 0.25% |
| CD105 | 97.68% | 98.28% | 98.25% | 97.32% |
| HLA-DR | 0.07% | 0.06% | 1.87% | 0.05% |

IV. Identification of Paracrine Capacity of Different Cryopreservation Preparations of hUC-MSCs

Stem cell stock solution from three different umbilical cord-deriveds (respectively numbered Y200001, Y200003, and Y210001) were taken as examples, the cryopreserved preparations of mesenchymal stem cells were prepared according to the different cryopreservation solutions listed in step 1 at a density of 5E6/mL. After the cryopreserved cells were thawed, the cryopreserved cells were inoculated into well plates at 20000/cm², and cultured in an incubator with 5% CO₂ and 37° C. After culturing for 48 hours, centrifuged to collect the supernatant, and ELISA kits were used to detect the contents of IL-6, HGF, MMP1 and MMP2 in the supernatant respectively.

The results were shown in Table 6. The results showed that: different cryopreserved preparations of mesenchymal stem cells prepared by the method of the present invention had the capacity to secrete IL-6, HGF, MMP1 and MMP2, thereby performing immune regulation.

TABLE 6
The capacity of different cryopreserved preparations of mesenchymal
stem cells to secrete IL-6, HGF, MMP1 and MMP2

| | | IL6 | HGF | MMP1 | MMP2 |
|---|---|---|---|---|---|
| | | secretion | secretion | secretion | secretion |
| | | capacity | capacity | capacity | capacity |
| Group | Cell source | (pg/mL ) | (pg/mL) | (pg/mL ) | (pg/mL) |
| C1 | Y200001 | 169.23 | 366.25 | 93990.64 | 18377.84 |
| | Y200003 | 131.30 | 2375.01 | 66995.44 | 21023.2 |
| | Y210001 | 375.32 | 1529.81 | 86204.37 | 23519.53 |
| C2 | Y200001 | 31.28 | 1603.06 | 101452.04 | 13776.39 |
| | Y200003 | 60.91 | 3172.32 | 97260.24 | 23221.46 |
| | Y210001 | 96.89 | 2730.00 | 124223.99 | 19793.67 |
| C3 | Y200001 | 24.83 | 2752.53 | 108075.08 | 13664.62 |
| | Y200003 | 24.83 | 3211.76 | 110317.70 | 21256.07 |
| | Y210001 | 46.44 | 3003.28 | 103893.76 | 16440.39 |
| C4 | Y200001 | 17.99 | 2400.37 | 112057.29 | 12081.12 |
| | Y200003 | 20.63 | 3879.47 | 106807.06 | 24255.39 |
| | Y210001 | 29.04 | 3059.62 | 111480.92 | 17837.59 |

V. Expression Levels of Active Factor mRNA in Different Cryopreservation Preparations of hUC-MSCs

Stem cell stock solution from three different umbilical cord-deriveds (respectively numbered Y200001, Y200003, and Y210001) were taken as examples, the cryopreserved preparations of mesenchymal stem cells were prepared according to the different cryopreservation solutions listed in step 1 at a density of 5E6/mL. After the cryopreserved cells were thawed, the cryopreserved cells were inoculated into well plates at 20000/cm² and cultured in serum-free complete medium (normal culture group) and serum-free complete medium (inflammation stimulation group) added with IFN-γ (final concentration 15 ng/mL) respectively. After culturing for 24 h, total RNA was extracted using an RNA extraction kit (TaKaRa, catalog number: 9767), and then the obtained total RNA was reverse-transcribed into cDNA using a reverse transcription kit (Thermo Fisher Scientific, catalog number K1621), and then obtained cDNA was used as template for QPCR. The primer sequences were respectively as follows:

Thermo Fisher (China) Co., Ltd.,

CXCL10-F:
(SEQ ID NO: 1)
5′-GGTGAGAAGAGATGTCTGAATCC-3′;
CXCL10-R:
(SEQ ID NO: 2)
5′-GTCCATCCTTGGAAGCACTGCA-3′;
CCL2-F:
(SEQ ID NO: 3)
5′-GCTGTAAGGACATCGCCTACCA-3′;
CCL2-R:
(SEQ ID NO: 4)
5′-AGAATCACCAGCAGCAAGTGTCC-3′;
IDO-F:
(SEQ ID NO: 5)
5′-GCCTGATCTCATAGAGTCTGGC-3′;
IDO-R:
(SEQ ID NO: 6)
5′-TGCATCCCAGAACTAGACGTGC-3′.

The QPCR system was as follows: upstream primer 0.4 μL; downstream primer 0.4 μL; Premix Ex Taq polymerase 10 μL; cDNA 2 μL; double-distilled water 7.2 μL.

The QPCR procedure was as follows:
1) 95° C. for 30 seconds, 1 cycle;
2) 95ºC for 5 seconds and 60° C. for 30 seconds, 40 cycles;
3) 95° C. for 5 seconds, 60° C. for 1 minute, 95° C. for 1 cycle
4) 50° C. for 30 seconds and 1 cycle.

The results were shown in FIG. 4-FIG. 6. The results showed that the inflammatory environment could induce significant up-regulation of the mRNA expression levels of functional factors CCL2, CXCL10 and IDO1 in MSCss. It showed that the cryopreserved preparation of mesenchymal stem cells prepared by the method of the present invention had good functions of immune regulation and promoting damage repair.

VI. Safety Testing of Cryopreservation Preparations of hUC-MSCs

### 1. Experimental Materials

Test product C1: the cryopreserved preparation of mesenchymal stem cells was prepared according to the method in step 1, the cell cryoprotectant was C1, and the cell concentration was 1.25×10⁷ cells/mL.

Solvent of test product: cell cryoprotectant C1.

Laboratory animals: 40 SPF grade CD-1 mice aged 6-7 weeks, half male and half female, provided by Zhejiang Vital River Laboratory Animal Technology Co., Ltd., and raised in SPF grade animal room of JOINN Laboratories (Suzhou) Co., Ltd, after adaptive feeding for 1 week, it was used for safety testing experiment.

### 2. Experimental Method

The mice were randomly divided into 2 groups according to body weight, and the dosage of each group was shown in Table 7. After grouping, all animals were given a single tail vein injection of 0.4 mL of corresponding test product C1 or solvent of test product, and the acute toxic reaction was observed by the side of the cage for at least 4 hours after administration, and then body weight and food intake were measured every week. After 14 days, anatomy and gross observation were performed, and abnormal tissues were examined by histopathology.

TABLE 7
Experimental grouping and administration dosage

| | | Dose | Administration | | ) Number of | |
|---|---|---|---|---|---|---|
| | | (cells/ | dosage | Concentration | animals(only) | |
| Groups | | only) | (mL/only) | (cells/mL) | Female | Male |
| 1 | Solvent | 0 | 0.4 | 0 | 10 | 10 |
| | control | | | | | |
| 2 | Test | 5 × 10⁶ | 0.4 | 1.25 × 10⁷ | 10 | 10 |
| | product | | | | | |
| | C1 | | | | | |

### 3. Experimental Results

During the experiment, none of the animals were found dead or dying. 1 male animal in group C1 of the test product showed purplish red at the distal end of the tail 2-9 days after administration, which was considered to be caused by mechanical damage during the administration and had nothing to do with the test product; 2 male animals had tail scabs 7-14 days after administration; 3 male animals had scrotal scabs 7-14 days after administration. Since the above animals were kept in the same cage, and the symptoms of scabs appeared at the same time, it may be caused by animal fights. It had nothing to do with the test product.

During the experiment, compared with the solvent control group, the body weight growth of male animals in the test product C1 group decreased in the first week (P<0.05, vs. vehicle control group), and the abnormality recovered in the second week. Saw Table 8 and Table 9 for details.

TABLE 8
Body weight and body weight growth of male mice in each group (g,
n = 10, Mean ± SD)

| | Weight (g) | | | Weight growth | |
|---|---|---|---|---|---|
| Group | 0 w | 1 w | 2 w | 0 w→1 w | 1 w→2 w |
| Solvent | 35.10 ± | 37.68 ± | 39.44 ± 2.76 | 2.58 ± 0.96 | 4.34 ± 1.36 |
| control | 1.75 | 2.58 | | | |
| Test | 35.21 ± | 36.53 ± | 38.96 ± 2.26 | 1.32 ± 0.82* | 3.75 ± 1.07 |
| product C1 | 1.69 | 1.81 | | | |

Note:
*P < 0.05 compared with solvent control group.

TABLE 9
Body weight and body weight growth of female mice in each group (g,
n = 10, Mean ± SD)

| | Weight (g) | | | Weight growth | |
|---|---|---|---|---|---|
| Group | 0 w | 1 w | 2 w | 0 w→1 w | 1 w→2 w |
| Solvent | 27.44 ± | 28.00 ± | 28.46 ± 1.25 | 0.56 ± 0.85 | 1.02 ± 1.00 |
| control | 0.96 | 0.84 | | | |
| Test | 27.56 ± | 27.97 ± | 28.16 ± 1.71 | 0.41 ± 1.12 | 0.60 ± 0.93 |
| product C1 | 1.04 | 1.74 | | | |

During the experiment, compared with the solvent control group, the food intake of female and male animals W1 in the test product C1 group decreased, which may be related to the test product, and the abnormality recovered in the second week. In addition, the food intake of male animals W2 also decreased slightly, so it was considered not to have toxicological significance. The results were specifically shown in Table 10.

TABLE 10
Food intake of mice in each group (g/only/day, n = 10, Mean ± SD)

| | 0 w→1 w | | 1 w→2 w food intake | |
|---|---|---|---|---|
| | food intake (g/only/day) | | (g/only/day) | |
| Groups | Male | Female | Male | Female |
| Solvent control | 6.28 ± 0.08 | 4.46 ± 0.16 | 6.11 ± 0.01 | 4.24 ± 0.05 |
| Test product C1 | 5.76 ± 0.12* | 4.16 ± 0.18* | 5.97 ± 0.11* | 4.28 ± 0.08 |

Note:
*P < 0.05 compared with solvent control group.

All animals were euthanized at the end of the administration period (D15), and no gross lesions related to the test product were found, so tissue preservation and microscopic observation were not performed.

To sum up, the cryopreservation preparation of hUC-MSCs

(test product C1) was given to CD-1 mice by single intravenous injection of 5.0×10⁶ cells/only, and no animals died, and no toxic reactions related to the test product were observed. Under the conditions of this experiment, the maximum tolerated dose of the test product C1 for mice was 5.0×10⁶ cells/mouse.

### Example 3. Use of Cryopreservation Preparation of hUC-MSCs in the Treatment of Novel Coronavirus Pneumonia (COVID-19)

### 1. Experimental Materials

Test product C1: the cryopreserved preparation of mesenchymal stem cells was prepared according to the method in step 2, containing cell cryoprotectant C1, with cell concentration of 1×10⁵ cells/mL, 1×10⁶ cells/mL, and 1×10⁷ cells/mL, respectively.

Solvent of test product: cell cryoprotectant C1.

Laboratory animals: 9 female hACE2-KI/NIFDC humanized mice (hACE2 mice) of SPF grade aged 7-8 weeks, provided by the National Institutes for Food and Drug Control, they were raised in the P4 positive pressure IVC breeding room of the Harbin Veterinary Research Institute of the Chinese Academy of Agricultural Sciences.

### 2. Experimental Method

The laboratory animals were randomly divided into 3 groups according to their body weight, namely the model control group, the C1 low-dose group and the C1 high-dose group, with 3 animals in each group. The specific groupings were shown in Table 11.

TABLE 11
Animal grouping and dose design

| | | | | | Administration |
|---|---|---|---|---|---|
| | | Number of | Dosage | Concentration | dosage |
| Groups | | animals(only) | (cells/only) | (cells/ mL) | (mL/only) |
| 1 | Model | 3 | 0 | 0 | 0.1 |
| | control | | | | |
| | group | | | | |
| 2 | C1 low- | 3 | 1 × 10⁵ | 1 × 10⁶ | 0.1 |
| | dose | | | | |
| 3 | C1 high- | 3 | 1 × 10⁶ | 1 × 10⁷ | 0.1 |
| | dose | | | | |

After grouping, all animals were lightly anesthetized with carbon dioxide, and 5×10⁵ PFU of SARS-COV-2 (HRB26) was given according to 50 μL/intranasal drops (SARS-COV-2 (HRB26) was recorded in the literature “Wang J, Shuai L, Wang C, et al. Mouse-adapted SARS-COV-2 replicates efficiently in the upper and lower respiratory tract of BALB/c and C57BL/6J mice[J]. Protein & cell, 2020, 11(10):776-782.”), 0 dpi on the day of exposure. The drug treatment was carried out at 1 dpi and 4 dpi respectively. Each administration group was given the corresponding dose of the test product C1 by tail vein injection according to Table 11, and the model control group was given the same amount of the solvent of test product.

The general condition of the animals was observed every day during the administration period. Animal body weight was measured once a day. All animals were euthanized at 5 dpi, and part of the lungs were taken for lung viral load determination (RT-qPCR). The remaining lungs (with part of the bronchi) were fixed in 4% paraformaldehyde, and pathologically observed by HE staining.

### 3. Experimental Results

Animals in each group did not die before and after the challenge, and no adverse reactions related to the test product C1 were seen.

The body weight changes of the low and high-dose groups of test product C1 were consistent with those of the model group, and both showed a downward trend at 2 dpi; the body weight of the administration group was different from the model control group at some time points, which may be related to the differences in body weight of each group before the challenge, the test product C1 had no significant effect on the state, body weight and weight growth of the model mice (Table 12).

TABLE 12
Effect of test product C1 on weight growth of COVID-19
mice (n = 3, Mean ± SD)

| time | Quantity | Model control | Low-dose | High-dose |
|---|---|---|---|---|
| (dpi) | (only) | group | group | group |
| 0 | 3 | 22.07 ± 0.15 | 21.23 ± 0.25* | 22.63 ± 0.31 |
| 1 | 3 | 22.33 ± 0.15 | 21.43 ± 0.23* | 22.77 ± 0.25 |
| 2 | 3 | 21.43 ± 0.25 | 21.33 ± 0.23 | 22.03 ± 0.21 |
| 3 | 3 | 21.40 ± 0.20 | 21.10 ± 0.00* | 22.43 ± 0.21* |
| 4 | 3 | 21.60 ± 0.26 | 21.23 ± 0.06* | 23.00 ± 0.20* |
| 5 | 3 | 21.70 ± 0.26 | 21.33 ± 0.06* | 22.53 ± 0.06* |

Note:
*P < 0.05 compared with the model control group.

Compared with the model control group, there was no statistical difference in the viral load in the lungs between the low-dose and high-dose groups of the test product C1 (P>0.05). The group found that the viral load in the lungs of 2 mice (3F02 and 3F03) decreased by about (1˜2 log 10 (copies/g)) (Table 13), suggesting that the test product C1 had a certain effect on the viral load in the lungs of the COVID-19 model mice.

TABLE 13
Effect of test product C1 on viral load in lung tissue of COVID-19
mice (n = 3, Mean ± SD)

| | Viral load in the | | |
|---|---|---|---|
| | lungs | x ± SD | Animal number |
| Groups | log₁₀ (copies/g) | log₁₀ (copies/g) | (♀) |
| Solvent control | 8.37 | 7.86 ± 0.71 | 1F01 |
| group | 7.05 | | 1F02 |
| | 8.16 | | 1F03 |
| Low-dose group | 8.26 | 5.48 ± 4.74 | 2F01 |
| | Negative | | 2F02 |
| | 8.17 | | 2F03 |
| High-dose group | 8.42 | 7.19 ± 1.09 | 3F01 |
| | 6.34 | | 3F02 |
| | 6.82 | | 3F03 |

Inflammatory infiltration around small blood vessels: the inflammatory infiltration around small blood vessels in the model control group was mild to moderate; the degree of inflammatory infiltration in the low-dose group of test product C1 had a tendency to improve, ranging from slight to light moderate; the inflammatory infiltration in the high-dose group of test product C1 was significantly improved, ranging from negative to mild; it suggested that the test product C1 could improve the inflammatory infiltration around small pulmonary vessels of the lung in a dose-dependent manner (Table 14).

Inflammatory infiltration around the bronchioles: the inflammatory infiltration around the bronchioles in the model control group was mild to moderate; the inflammatory infiltration in the low-dose group of test product C1 had a tendency to improve, ranging from negative to mild; the inflammatory infiltration in the high-dose group of test product C1 was significantly improved, ranging from negative to mild; it suggested that the test product C1 could improve the inflammatory infiltration around the bronchioles of the lung in a dose-dependent manner (Table 14).

Degeneration of bronchial epithelial cells: the degeneration of bronchiole epithelial cells in the model control group was slight to mild; the degeneration of epithelial cells in the low-dose group of test product C1 was significantly improved, ranging from negative to mild; the high-dose group of test product C1 was slight to mild; it suggested that the test product C1 had a significant improvement effect on the degeneration of lung bronchiole epithelial cells (Table 14).

TABLE 14
Effect of test product C1 on lung histopathology in COVID-19 mice

| | | Animal |
|---|---|---|
| Groups | Pathological results of lung tissue | number |
| Model | Moderate inflammatory cell infiltration around small | 1F01 |
| control | blood vessels, moderate inflammatory cell | |
| group | infiltration around bronchioles, mild degeneration of | |
| | bronchiole epithelial cells | |
| | Mild inflammatory cell infiltration around small | 1F02 |
| | blood vessels, mild inflammatory cell infiltration | |
| | around bronchioles, slight degeneration of bronchiole | |
| | epithelial cells | |
| | Mild inflammatory cell infiltration around small | 1F03 |
| | blood vessels, mild inflammatory cell infiltration | |
| | around bronchioles, slight degeneration of bronchiole | |
| | epithelial cells | |
| Low- | Mild inflammatory cell infiltration around small | 2F01 |
| dose | blood vessels, mild inflammatory cell infiltration | |
| group | around bronchioles | |
| of test | Mild inflammatory cell infiltration around small | 2F02 |
| product | blood vessels, mild inflammatory cell infiltration | |
| C1 | around bronchiole, mild local inflammatory cell | |
| | infiltration | |
| | Mild-moderate inflammatory cell infiltration around | 2F03 |
| | small blood vessels, mild degeneration of bronchiole | |
| | epithelial cells | |
| High- | Mild inflammatory cell infiltration around small | 3F01 |
| dose | blood vessels, mild inflammatory cell infiltration | |
| group of | around bronchioles, mild degeneration of bronchiole | |
| test | epithelial cells | |
| product | Mild inflammatory cell infiltration around small | 3F02 |
| C1 | blood vessels, mild degeneration of bronchiolar | |
| | epithelial cells | |
| | Mild inflammatory cell infiltration around blood | 3F03 |
| | vessels, slight degeneration of bronchiolar epithelial | |
| | cells | |

In summary, the test product C1 could improve the viral load in the lungs and lung histopathology of COVID-19 mice, among which the improvement of inflammatory infiltration around small blood vessels and bronchioles was the most significant, and the effective dose was 1×10⁵ cells/only.

### Example 4. Use of Cryopreservation Preparation of hUC-MSCs in the Treatment of Idiopathic Pulmonary Interstitial Fibrosis

### 1. Experimental Materials

Test product C1: the cryopreserved preparation of mesenchymal stem cells was prepared according to the method in step 1 of Example 2, containing cell cryoprotectant C1, with cell concentrations of 1×10⁶ cells/mL, 3×10⁶ cells/mL, and 1×10⁷ cells/mL, respectively.

Solvent of test product: cell cryoprotectant C1.

Laboratory animals: 82 male SD rats of SPF grade aged 7-10 weeks, provided by Zhejiang Vital River Laboratory Animal Technology Co., Ltd., and raised in the SPF grade animal room of JOINN Laboratories (Suzhou) Co., Ltd.

### 2. Experimental Method

The laboratory animals were divided into normal control group (10 rats) and model group (72 rats) according to body weight. On day 1 (D1) and day 3 (D3) after grouping, rats in model group were administered via intratracheal inhalation with 2.5 mg/kg (on D1) and 1 mg/kg (on D3) of bleomycin (BLM) to build pulmonary fibrosis (PF) model, and rats in normal control group were administered with sodium chloride injection. After the second modeling, the animals in model group were randomly divided into model control group, C1 low-dose group, C1 medium-dose group, and C1 high-dose group again according to body weight, with 10 animals in each group. The specific groups were shown in Table 15.

TABLE 15
Animal grouping and dose design

| | | Number | | | Admin- |
|---|---|---|---|---|---|
| | | of | | Concen- | istration |
| | | animals | Dosage | tration | dosage |
| Groups | | (only) | (cells/kg) | (cells/ mL) | (mL/kg) |
| 1 | Normal control | 10 | 0 | 0 | 1 |
| | group | | | | |
| 2 | Model control | 10 | 0 | 0 | 1 |
| | group | | | | |
| 3 | C1 low-dose | 10 | 1 × 10⁶ | 1 × 10⁶ | 1 |
| 4 | C1 medium- | 10 | 3 × 10⁶ | 3 × 10⁶ | 1 |
| | dose | | | | |
| 5 | C1 high-dose | 10 | 1 × 10⁷ | 1 × 10⁷ | 1 |

Rats in C1 low-dose group, C1 medium-dose group, and C1 high-dose group were intravenously administered with different doses of the test product intail vein on D4, D7 and D10 respectively (saw Table 15 for the specific dose), and rats in normal control group and model control group were intravenously administered with the solvent of test product (cell cryoprotectant C1) in lateral tail vein on D4, D7 and D10 respectively.

After the rats were anesthetized on D28, dynamic lung compliance (Cdyn), airway resistance (RL), forced vital capacity (FVC) and other indicators were detected using the pulmonary function analysis system. Then the whole lung tissue was taken out and weighed to calculate pulmonary index. After the lung tissue was weighed, the left lung tissue was taken to detect the content of hydroxyproline (HYP), MMP-2, and TIMP-1. The remaining right lung tissue and bronchi were fixed for histopathological examination.

### 3. Experimental Results

After BLM modeling, the lung weight and pulmonary index of rats in model control group significantly increased (P<0.01), and the pulmonar index of PF (pulmonary fibrosis) model rats in each dose group of the test product decreased (P<0.01) (Table 16).

TABLE 16
The effect of the test product on pulmonary index of
BLM-induced PF model rats (n = 10, Mean ± SD)

| | Drug | Body | Lung | Pulmonary |
|---|---|---|---|---|
| | dosage | weight | weight | index |
| Groups | (cells/kg) | (g) | (g) | (%) |
| Normal | — | 453.30 ± | 1.99 ± | 0.437 ± |
| control | | 36.69** | 0.33** | 0.056** |
| group | | | | |
| Model | — | 290.90 ± | 3.52 ± | 1.209 ± |
| control | | 22.18 | 0.47 | 0.129 |
| group | | | | |
| C1 low- | 1 × 10⁶ | 367.40 ± | 3.24 ± | 0.907 ± |
| dose group | | 59.87** | 0.40 | 0.204** |
| C1 medium- | 3 × 10⁶ | 404.60 ± | 3.07 ± | 0.765 ± |
| dose group | | 55.33** | 0.38 | 0.073** |
| C1 high- | 1 × 10⁷ | 372.60 ± | 3.15 ± | 0.854 ± |
| dose group | | 48.99** | 0.55 | 0.160** |

Note:
Compared with the model control group, **P < 0.01.

After BLM-induced modeling, the Cdyn and FVC of rats in control group were significantly lower than those in normal control group, and RL was significantly higher. FVC of model rats in each C1 dose group significantly increased, wherein Cdyn and RL of model rats in low-dose group significantly increased, and Cdyn of model rat in high-dose group also significantly increased. It was suggested that the test product C1 could reduce the lung function damage caused by fibrosis (Table 17).

TABLE 17
The effect of the test product C1 on lung function of
BLM-induced PF model rats (n = 10, Mean ± SD)

| | Drug | Cdyn | | |
|---|---|---|---|---|
| | dosage | (mL/ | RL | FVC |
| Groups | (cells/kg) | cmH₂O) | (cmH₂O) | (mL) |
| Normal | — | 0.25 ± | 0.24 ± | 12.81 ± |
| control | | 0.06** | 0.06** | 1.61** |
| group | | | | |
| Model | — | 0.14 ± | 0.45 ± | 5.48 ± |
| control | | 0.03 | 0.09 | 1.18 |
| group | | | | |
| C1 low-dose | 1 × 10⁶ | 0.22 ± | 0.30 ± | 8.52 ± |
| group | | 0.06** | 0.08** | 1.93** |
| C1 medium- | 3 × 10⁶ | 0.19 ± | 0.39 ± | 7.86 ± |
| dose group | | 0.07 ^{p = 0.07} | 0.20 | 2.15** |
| C1 high- | 1 × 10⁷ | 0.22 ± | 0.35 ± | 8.87 ± |
| dose group | | 0.10* | 0.18 | 2.90** |

Note:
Compared with the model control group, **P < 0.01, *P < 0.05.

After BLM modeling, HYP, MMP-2, and TIMP-1 in the lung tissue of control group were significantly higher than those of normal control group. HYP and TIMP-1 of PF rats in each dose group of C1 significantly decreased, and the overexpression of MMP-2 in low and medium dose groups of C1 was also significantly inhibited. It was suggested that the test product C1 could inhibit the expression of fibrosis-promoting factors, improve collagen deposition, and play an anti-PF role (Table 18).

TABLE 18
The effect of the test product C1 on the fibrosis-related indicators
of BLM-induced PF model rats (n = 10, Mean ± SD)

| | Drug | | | |
|---|---|---|---|---|
| | dosage | HYP | MMP-2 | TIMP-1 |
| Groups | (cells/kg) | (mg/mL) | (ng/ml) | (pg/mL) |
| Normal | — | 0.41 ± | 39.01 ± | 2524.91 ± |
| control | | 0.03** | 10.91 ^{p = 0.07} | 850.49** |
| group | | | | |
| Model | — | 0.60 ± | 45.82 ± | 3618.23 ± |
| control | | 0.14 | 0.78 | 94.10 |
| group | | | | |
| C1 low- | 1 × 10⁶ | 0.48 ± | 44.76 ± | 3472.98 ± |
| dose group | | 0.08* | 0.63** | 219.16 ^{p = 0.07} |
| C1 | 3 × 10⁶ | 0.49 ± | 43.73 ± | 3408.82 ± |
| medium- | | 0.05* | 0.72** | 214.32** |
| dose group | | | | |
| C1 high- | 1 × 10⁷ | 0.48 ± | 41.46 ± | 3424.00 ± |
| dose group | | 0.07* | 11.56 | 110.54** |

Note:
Compared with the model control group, **P < 0.01, *P < 0.05.

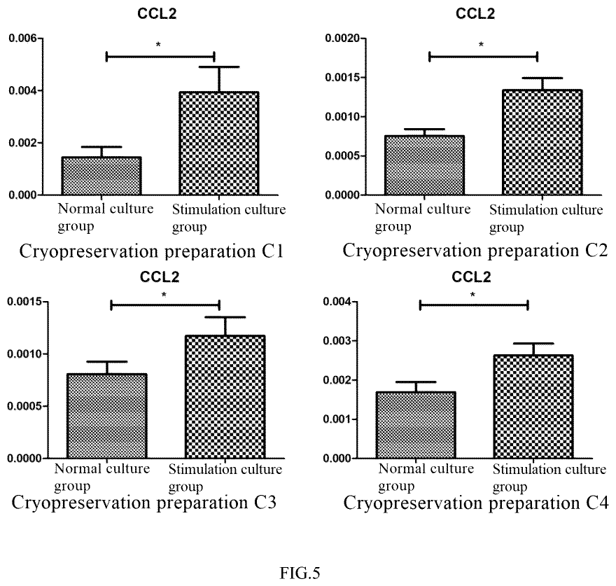

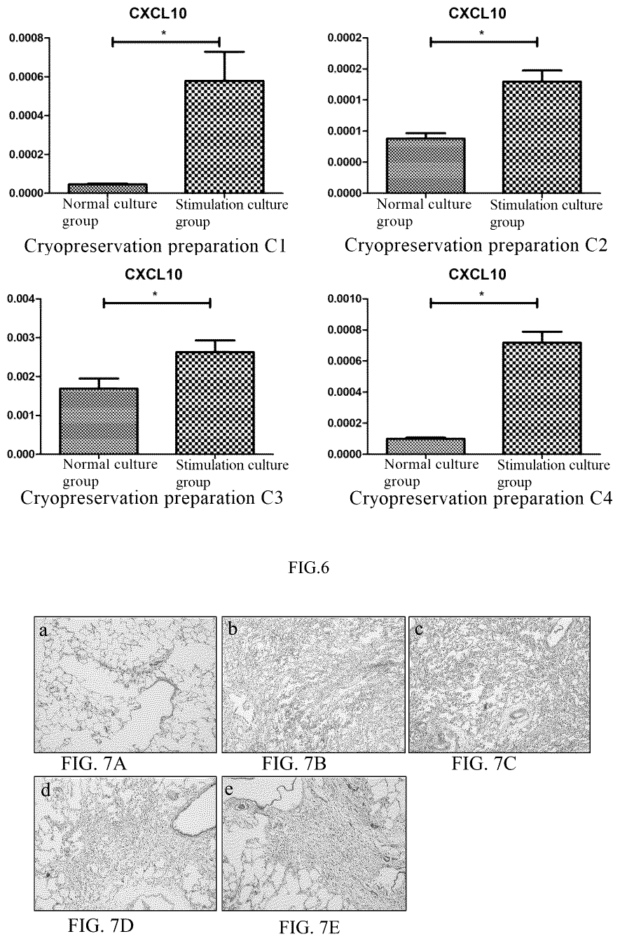

After BLM modeling, in model control group, moderate to severe multifocal inflammatory cell infiltration occurred in the alveoli and moderate to severe multifocal fibrosis occurred in the interstitium/alveoli of the lungs, accompanied by different degrees of multifocal alveolar dilation, macrophage aggregation, hemorrhage/congestion, and fibrinous exudate. C1 could dose-dependently improve pulmonary interstitial/trachea/perivascular/alveolar inflammatory infiltration and fibrosis, among which high-dose C1 showed the most significant improvement (Table 19, FIG. 7).

TABLE 19
Effect of the test product C1 on lung histopathology
of BLM-induced PF model rats (n = 10)

| | Number of animals with | | | | | | | | | |
|---|---|---|---|---|---|---|---|---|---|---|
| | inflammatory cell | | | | | Number of animals with | | | | |
| | infiltration of each grade | | | | | fibrosis of each grade | | | | |
| Groups | − | + | ++ | +++ | ++++ | − | + | ++ | +++ | ++++ |
| Normal | 10 | 0 | 0 | 0 | 0 | 10 | 0 | 0 | 0 | 0 |
| control group | | | | | | | | | | |
| Model | 0 | 0 | 0 | 0 | 10 | 0 | 0 | 0 | 0 | 10 |
| control group | | | | | | | | | | |
| C1 low-dose | 0 | 0 | 1 | 8 | 1 | 0 | 0 | 0 | 7 | 3 |
| C1 medium- | 0 | 0 | 2 | 8 | 0 | 0 | 0 | 0 | 10 | 0 |
| dose | | | | | | | | | | |
| C1 high-dose | 0 | 2 | 7 | 1 | 0 | 0 | 0 | 8 | 2 | 0 |

Note:
“−” no lesion, “+” slight; “++” mild; “+++” moderate; “++++” obvious; “+++++” severe.

In summary, the test product C1 could improve the collagen deposition in lung tissue by inhibiting the expression of fibrosis-promoting factors, and significantly improve the lung function damage, lung inflammation and fibrosis caused by BLM. It was suggested that the test product C1 had a better anti-pulmonary fibrosis effect at an effective dose of 1×10⁶ cells/kg in rats.

### Example 5. Use of Cryopreservation Preparation of hUC-MSCs in the Treatment of Acute Lung Injury

### 1. Experimental Materials

Test product C1: the cryopreserved preparation of mesenchymal stem cells was prepared according to the method in step 1 of Example 2, containing cell cryoprotectant of C1, with concentrations of 1×10⁶ cells/mL, 3×10⁶ cells/mL, and 1×10⁷ cells/mL, respectively.

Solvent of test product: cell cryoprotectant C1.

Laboratory animals: 70 male SD rats of SPF grade aged 6-8 weeks, provided by Zhejiang Vital River Laboratory Animal Technology Co., Ltd., and raised in the SPF grade animal room of JOINN Laboratories (Suzhou) Co., Ltd.

### 2. Experimental Method

The 70 male rats that passed the quarantine were randomly divided into 5 groups according to body weight, saw Table 20 for details.

TABLE 20
Animal grouping and dose design

| | | Number | | | Admin- |
|---|---|---|---|---|---|
| | | of | | Concen- | istration |
| | | animals | Dosage | tration | dosage |
| Groups | | (only) | (cells/kg) | (cells/ mL) | (mL/kg) |
| 1 | Normal | 10 | 0 | 0 | 1 |
| | control group | | | | |
| 2 | Model | 10 | 0 | 0 | 1 |
| | control group | | | | |
| 3 | C1 low-dose | 10 | 1 × 10⁶ | 1 × 10⁶ | 1 |
| 4 | C1 medium- | 10 | 3 × 10⁶ | 3 × 10⁶ | 1 |
| | dose | | | | |
| 5 | C1 high-dose | 10 | 1 × 10⁷ | 1 × 10⁷ | 1 |

On day 1 (D1) and day 3 (D3) after grouping, rats in model control group and each C1 dose group were administered via intratracheal inhalation with 5 mg/kg (on D1) and 0.8 mg/kg (on D3) of lipopolysaccharide (LPS) to build an acute lung injury (ALI) model, and rats in the normal control group were administered with sodium chloride injection. D1 and D3 were given LPS for 4-6 hours, and then administered different doses of the test product by tail vein according to Table 20. The normal control group and the model control group were given the same amount of the solvent of test product.

After D4 rats were anesthetized, 0.2 mL of arterial blood was collected for blood gas analysis. Subsequently, 2 mL of alveolar lavage fluid was collected, centrifuged and resuspended in 1 mL of PBS to settle to the bottom, and white blood cells and differential counts were performed with an automatic hematology analyzer. The middle lobe tissue of the right lung of the animal was taken and weighed, then placed in an oven at 60° C. for 72 hours and weighed again to calculate the wet/dry weight ratio. The remaining right lung tissue and bronchi were fixed in 10% neutral buffered formalin solution for pathological examination.

The experimental data were expressed as “mean #standard deviation”. Statistical software SPSS 13.0 and/or GraphPad Prism 5 were used to process the data, and P<0.05 was considered statistically significant.

### 3. Experimental Results

C1 could dose-dependently reduce the lung wet-to-dry weight ratio of ALI rats, and there was a significant difference between the medium and high-dose groups of C1 and the model control group; suggesting that the test product C1 could reduce the lung water content and improve pulmonary edema (Table 21).

TABLE 21
Effect of test product C1 on lung water content of
LPS-induced ALI model rats (n = 10, Mean ± SD)

| Groups | Drug dosage | Lung wet-to-dry ratio |
|---|---|---|
| Normal control group | — | 4.33 ± 0.19** |
| Model control group | — | 5.17 ± 0.22 |
| C1 low-dose group | 1 × 10⁶ cells/kg | 4.95 ± 0.29^{P = 0.07} |
| C1 medium-dose | 3 × 10⁶ cells/kg | 4.91 ± 0.19* |
| group | | |
| C1 high-dose group | 1 × 10⁷ cells/kg | 4.64 ± 0.18** |

Note:
Compared with the model control group, **P < 0.01, *P < 0.05.

Each group of C1 could significantly increase the PCO2 of the rats in the model control group, and the PO2 and sO2 in the low-dose group and the PO2 in the medium-dose group were also significantly higher than those of the model control group; suggesting that the test product C1 could improve the acute respiratory distress syndrome (ARDS) of ALI rats (Table 22).

TABLE 22
The effect of the test product C1 on the results of arterial blood gas
analysis in LPS-induced ALI model rats (n = 10, Mean ± SD)

| Groups | PO₂ | sO₂ | PCO₂ | pH |
|---|---|---|---|---|
| Normal | 98.44 ± | 97.11 ± | 45.78 ± | 7.38 ± |
| control | 11.47** | 1.05* | 7.99** | 0.06** |
| group | | | | |
| Model | 77.10 ± | 95.70 ± | 33.32 ± | 7.45 ± |
| control | 8.12 | 1.57 | 6.52 | 0.04 |
| group | | | | |
| C1 | 93.00 ± | 97.30 ± | 39.71 ± | 7.44 ± |
| low-dose | 10.90** | 1.16* | 4.33* | 0.03 |
| group | | | | |
| C1 | 88.40 ± | 96.60 ± | 42.18 ± | 7.43 ± |
| medium- | 10.62* | 1.43 | 4.33** | 0.03 |
| dose group | | | | |
| C1 high- | 86.40 ± | 96.10 ± | 43.66 ± | 7.42 ± |
| dose group | 13.13 ^{P = 0.07} | 2.02 | 5.39** | 0.03 |

Note:
Compared with the model control group, **P < 0.01, *P < 0.05.

Each dose group of C1 significantly reduced the abnormal increase of total protein content, white blood cell count and differential count in the BALF of ALI rats, suggesting that the test product C1 had a significant improvement effect on lung inflammation (Table 23).

TABLE 23
Effect of test product C1 on total protein,
white blood cell and differential count in BALF
of ALI model rats (n = 10, Mean ± SD)

| | | White | | | |
|---|---|---|---|---|---|
| | | blood | Neutro- | Lympho- | Mono- |
| | Total | cell | phils | cytes | cytes |
| | protein | (×10⁹ | (×10⁹ | (×10⁹ | (×10⁹ |
| Groups | (pg/mL) | cells/L | cells/L | cells/L | cells/L |
| Normal | 0.15 ± | 0.12 ± | 0.02 ± | 0.09 ± | 0.00 ± |
| control | 0.02** | 0.07** | 0.02** | 0.05** | 0.00** |
| group | | | | | |
| Model | 0.67 ± | 9.05 ± | 5.68 ± | 2.26 ± | 0.77 ± |
| control | 0.28 | 4.28 | 2.56 | 1.60 | 0.62 |
| group | | | | | |
| C1 low- | 0.36 ± | 4.71 ± | 2.55 ± | 1.81 ± | 0.22 ± |
| dose | 0.06** | 1.59** | 1.27** | 1.28 | 0.19* |
| group | | | | | |
| C1 | 0.30 ± | 2.57 ± | 0.99 ± | 1.44 ± | 0.07 ± |
| medium- | 0.08** | 0.91** | 0.67** | 0.65 | 0.04** |
| dose | | | | | |
| group | | | | | |
| C1 high- | 0.24 ± | 1.99 ± | 1.14 ± | 0.68 ± | 0.11 ± |
| dose | 0.05** | 0.87** | 0.80** | 0.47** | 0.05** |
| group | | | | | |

Note: Compared with the model control group, **P < 0.01, *P < 0.05.

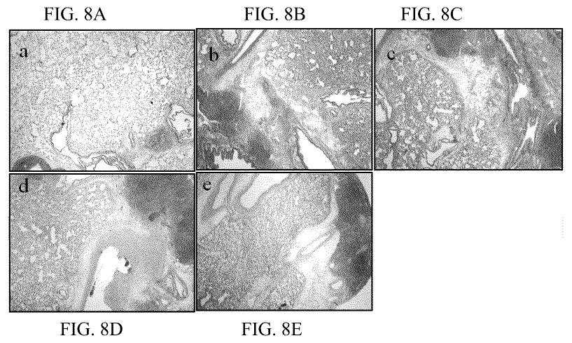

Interstitial/alveolar inflammatory cell infiltration, bronchi-associated lymphoid tissue hyperplasia, and alveolar hemorrhage could be seen in model animals. Each dose group of C1 could improve the lung tissue inflammatory infiltration and the lesion degree of lymphoid tissue hyperplasia of LPS-induced ALI model rats, suggesting that the test Product C1 had a certain therapeutic effect on pneumonia (Table 24, FIG. 8).

TABLE 24
The effect of test product C1 on the pathology
of lung tissue in ALI model rats (n = 10)

| | | Number | Interstitial/alveolar | | | | Bronchi-associated | | |
|---|---|---|---|---|---|---|---|---|---|
| | Dosage | of | inflammatory cell | | | | lymphoid | | |
| | (cells/ | animals | infiltration | | | | hyperplasia | | |
| Groups | kg) | (only) | − | + | ++ | +++ | − | + | ++ |
| Normal | — | 10 | 8 | 2 | 0 | 0 | 10 | 0 | 0 |
| control | | | | | | | | | |
| group | | | | | | | | | |
| Model | — | 10 | 0 | 0 | 4 | 6 | 1 | 6 | 3 |
| control | | | | | | | | | |
| group | | | | | | | | | |
| C1 low- | 1 × 10⁶ | 10 | 0 | 1 | 7 | 2 | 5 | 4 | 1 |
| dose | | | | | | | | | |
| group | | | | | | | | | |
| C1 | 3 × 10⁶ | 10 | 0 | 2 | 8 | 0 | 8 | 2 | 0 |
| medium- | | | | | | | | | |
| dose | | | | | | | | | |
| group | | | | | | | | | |
| C1 high- | 1 × 10⁷ | 10 | 0 | 4 | 6 | 0 | 8 | 2 | 0 |
| dose | | | | | | | | | |
| group | | | | | | | | | |

Note:
The degree of lesion “−” means normal; “+” means slight; “++” means mild; “+++” means moderate.

In summary, the test product C1 could significantly improve the lung inflammation, pulmonary edema and respiratory distress caused by LPS, suggesting that the test product C1 had a good anti-ALI effect, and the effective dose in rats was 1×10⁶ cells/kg.

### Example 6. Use of Cryopreservation Preparation of hUC-MSCs in the Treatment of Liver Fibrosis

1. Experimental Materials

Test product C1: the cryopreserved preparation of mesenchymal stem cells was prepared according to the method in step 2, containing cell cryoprotectant C1 with cell concentration of 1×10⁶ cells/mL, 5×10⁶ cells/mL, and 1.5×10⁷ cells/mL, respectively.

Solvent of test product: cell cryoprotectant C1.

Laboratory animals: 20 male SD rats of SPF grade aged 6-8 weeks, provided by provided by the Experimental Animal Center of the Academy of Military Medical Sciences of the Chinese People's Liberation Army, and raised in the animal room of Heze Biotechnology Co., Ltd.

### 2. Experimental Method

Rats qualified for quarantine were injected intraperitoneally with CCL4 twice a week for 8 consecutive weeks to establish a liver fibrosis model. After modeling, they were randomly divided into 4 groups, saw Table 25 for details.

TABLE 25
Experimental grouping and dose design

| | | Animals | Dosage |
|---|---|---|---|
| Groups | | (only) | (cells/only) |
| 1 | Model control group | 5 | 0 |
| 2 | C1 low-dose group | 5 | 1 × 10⁶ |
| 3 | C1 medium-dose group | 5 | 5 × 10⁶ |
| 4 | C1 high-dose group | 5 | 1.5 × 10⁷ |

After grouping, according to Table 25, a single tail vein injection of the corresponding dose of C1 was given, and the model control group was given equal PBS. One week later, all rats were given a modeling dose of CCL4 by intraperitoneal injection. At the second week after treatment, blood was collected, serum was separated, and four items of transaminase and liver fibrosis were detected. Then the animals were sacrificed, the liver was weighed, and the liver index was calculated. HE staining and Masson staining were used for liver histopathological detection.

### 3. Experimental Results

The test product C1 had a certain tendency to reduce the liver weight and liver coefficient of the liver fibrosis model rats, but there was no statistical difference compared with the model control group (Table 26).

TABLE 26
The effect of the test product C1 on the liver coefficient of
CCL₄-induced liver fibrosis model rats (n = 5, Mean ± SD)

| | | Lung weight | Liver coefficient |
|---|---|---|---|
| Groups | Drug dosage | (g) | (%) |
| Model control | — | 20.36 ± 4.41 | 3.82 ± 0.44 |
| group | | | |
| C1 low-dose | 1 × 10⁶ cells/only | 17.74 ± 2.85 | 3.33 ± 0.42 |
| group | | | |
| C1 medium- | 5 × 10⁶ cells/only | 18.63 ± 3.89 | 3.70 ± 0.60 |
| dose group | | | |
| C1 high-dose | 1.5 × 10⁷ cells/only | 19.60 ± 2.71 | 3.42 ± 0.25 |
| group | | | |

Each dose group of C1 had a certain improvement on the AST of the model rats, and the serum AST level of the medium-dose group of C1 was significantly lower than that of the model control group, but there was no obvious improvement on ALT (Table 27).

TABLE 27
The effect of test product C1 on liver function in
CCL₄-induced liver fibrosis model rats (n = 5, Mean ± SD)

| Groups | Dosage | ALT | AST |
|---|---|---|---|
| Model control | — | 47.42 ± | 126.14 ± |
| group | | 7.60 | 12.59 |
| C1 low-dose | 1 × 10⁶ cells/only | 46.30 ± | 102.24 ± |
| group | | 9.43 | 24.54^{p = 0.08} |
| C1 medium- | 5 × 10⁶ cells/only | 38.70 ± | 81.43 ± |
| dose group | | 9.95 | 25.92* |
| C1 high-dose | 1.5 × 10⁷ cells/only | 41.68 ± | 113.20 ± |
| group | | 13.82 | 18.24 |

Note:
*P < 0.05 compared with the model control group.

Each dose group of C1 had some improvement on the four indexes of liver fibrosis, among which the low and high dose groups of C1 could significantly reduce the content of HA and CIV in model rats, and the medium-dose group could significantly reduce the content of PCIII (Table 28).

TABLE 28
The effect of the test product C1 on the four items of liver fibrosis
in CCL₄-induced liver fibrosis model rats (n = 5, Mean ± SD)

| | Dosage | | | | |
|---|---|---|---|---|---|
| | (cells/ | HA | LN | PCIII | CIV |
| Groups | only) | (ng/mL) | (ng/mL) | (ng/mL) | (ng/mL) |
| Model | — | 182.70 ± | 28.22 ± | 27.30 ± | 7.74 ± |
| control | | 54.47 | 12.26 | 8.78 | 1.81 |
| group | | | | | |
| C1 low- | 1 × 10⁶ | 89.77 ± | 40.81 ± | 15.74 ± | 4.02 ± |
| dose | | 13.1** | 10.24 | 19.41 | 2.59* |
| group | | | | | |
| C1 | 5 × 10⁶ | 113.89 ± | 33.91 ± | 11.18 ± | 5.50 ± |
| medium- | | 46.14 | 12.82 | 5.99* | 2.87 |
| dose | | | | | |
| group | | | | | |
| C1 high- | 1.5 × 10⁷ | 108.36 ± | 31.08 ± | 15.40 ± | 2.65 ± |
| dose | | 34.57* | 16.33 | 14.04 | 1.53** |
| group | | | | | |

Note:
Compared with the model control group, **P < 0.01, *P < 0.05.

The rats in the model control group had disordered liver structure, and had mild fatty degeneration and thickened collagen fiber deposition; collagen regression was not obvious in the low-dose C1 group, and liver structure and collagen deposition in the medium and high-dose groups were significantly improved.

In summary, the test product C1 had a certain improvement effect on abnormal liver function and liver fibrosis caused by CCL4, and the effective dose of rats was 5×10⁶ cells/rat.

### Example 7. Use of Cryopreservation Preparation of hUC-MSCs in the Treatment of Crohn's Disease

### 1. Experimental Materials

Test product C1: the cryopreserved preparation of mesenchymal stem cells was prepared according to the method in step 1 of Example 2, containing cell cryoprotectant C1, with cell concentrations of 1×10⁶ cells/mL, 2×10⁶ cells/mL, 1×10⁷ cells/mL, and 2×10⁷ cells/mL, respectively.

Solvent of test product: cell cryoprotectant C1.

Laboratory animals: 46 male SD rats of SPF grade aged 6-8 weeks, provided by Beijing Vital River Laboratory Animal Technology Co., Ltd., and raised in the SPF grade animal room of Beijing Zhaoyan New Drug Research Center Co., Ltd.

### 2. Experimental Method

Rats that passed the quarantine were randomly divided into 6 groups according to body weight. The specific groupings were shown in Table 29.

TABLE 29
Animal grouping and dose design

| | | | | Admin- | |
|---|---|---|---|---|---|
| | Number | | Concen- | istration | [symbol] |
| | of | Dosage | tration | dosage | Mode of |
| | animals | (cells/ | (cells/ | (mL/ | admin- |
| Groups | (only) | only) | mL) | only) | istration |
| Normal | 6 | 0 | 0 | 1 | — |
| control group | | | | | |
| Model control | 8 | 0 | 0 | 1 | — |
| group | | | | | |
| C1 | 8 | 1 × 10⁶ | 1 × 10⁶ | 1 | Intra- |
| intraperitoneal | | | | | peritoneal |
| low dose | | | | | injection |
| C1 | 8 | 1 × 10⁷ | 1 × 10⁷ | 1 | Intra- |
| intraperitoneal | | | | | peritoneal |
| high dose | | | | | injection |
| C1 vein low | 8 | 1 × 10⁶ | 2 × 10⁶ | 0.5 | Tail vein |
| dose | | | | | injection |
| C1 vein high | 8 | 1 × 10⁷ | 2 × 10⁷ | 0.5 | Tail vein |
| dose | | | | | injection |

After grouping, the rats were administered dinitrobenzenesulfonic acid (DNBS, 3.0 mL/kg) in the colon to establish the Crohn's disease model, and the next day (D1) and the fifth day (D5) after modeling, the corresponding doses of the test product were administered according to Table 29, and the normal control group and the model control group were given the same amount of the solvent of test product.

After the DNBS model was established, the body weight was weighed every day, and the stool properties and bloody stool were observed and recorded, and the disease activity index (DAI) was evaluated according to Table 30. DAI=(weight loss score+stool consistency score+scoring blood in stool)/3. D6 all animals were euthanized, colons were collected and measured for length and ulceration area, and then colon tissues were fixed for histopathological examination.

TABLE 30
Criteria for Scoring Clinical Symptoms

| Score | Weight loss | Stool pattern | Blood in stool |
|---|---|---|---|
| 0 | □0 | Normal | Fecal occult blood |
| | | | negative |
| 1 | 0-5% | Mild loose | Positive for fecal |
| | | stool | occult blood |
| 2 | 5%-10% | Loose stool | Strong positive |
| | | | for fecal occult |
| | | | blood |
| 3 | 10%-20% | Watery stool | Mild blood in |
| | | | stool |
| 4 | >20% | Severe | Severe bloody |
| | | diarrhea | stool |

### 3. Experimental Results

After modeling, the weight gain rate of animals decreased significantly. Compared with the normal control group, the body weight of other groups decreased significantly on D1-D6, and there was no statistical difference among other groups (Table 31).

TABLE 31
The effect of test product C1 on the body weight of DNBS-induced Crohn's
disease model rats (n = 6-8, Mean ± SD)

| | Before | | | | | | |
|---|---|---|---|---|---|---|---|
| Groups | grouping | D 1 | D 2 | D 3 | D 4 | D 5 | D 6 |
| Normal | 238.17 ± | 264.2 ± | 277.3 ± | 286.5 ± | 298.3 ± | 306.5 ± | 316.8 ± |
| control group | 11.7 | 9.6 | 9.5 | 10.0 | 12.0 | 12.2 | 14.0 |
| Model control | 238.1 ± | 231.0 ± | 225.1 ± | 233.9 ± | 242.1 ± | 248.1 ± | 255.4 ± |
| group | 9.0 | 9.0 | 8.5 | 10.3 | 15.1 | 19.7 | 24.8 |
| C1 | 239.1 ± | 228.3 ± | 226.8 ± | 231.4 ± | 237.8 ± | 242.8 ± | 248.0 ± |
| intraperitoneal | 9.7 | 10.3 | 10.7 | 16.2 | 24.0 | 24.7 | 31.9 |
| low-dose group | | | | | | | |
| C1 | 235.3 ± | 228.1 ± | 221.9 ± | 224.0 ± | 231.5 ± | 241.0 ± | 252.2 ± |
| intraperitoneal | 8.3 | 9.9 | 16.7 | 18.6 | 16.4 | 9.9 | 22.0 |
| high-dose group | | | | | | | |
| C1 vein low- | 235.4 ± | 227.6 ± | 221.6 ± | 224.3 ± | 230.6 ± | 240.4 ± | 253.4 ± |
| dose group | 9.3 | 8.2 | 5.3 | 5.8 | 10.1 | 22.4 | 20.3 |
| C1 vein high- | 240.8 ± | 234.5 ± | 229.5 ± | 238.3 ± | 248.8 ± | 251.1 ± | 259.6 ± |
| dose group | 10.6 | 10.1 | 11.5 | 13.0 | 17.7 | 24.8 | 27.2 |

After modeling, the DAI scores in each group increased significantly, and then gradually decreased. The DAI scores in the intraperitoneal injection high-dose group and the intravenous injection low-dose group on D5 and D6 showed a significant decrease trend, and the intraperitoneal injection high-dose group on D5 decreased by 58.00% compared with the model control group, the intravenous injection low-dose group of D6 decreased by 33.33% compared with the model control group (Table 32).

TABLE 32
Effect of test product C1 on DAI score of DNBS-induced Crohn's
disease model rats (n = 6-8, Mean ± SD)

| Groups | D 1 | D 2 | D 3 | D 4 | D 5 | D 6 |
|---|---|---|---|---|---|---|
| Normal | 0.00 ± | 0.00 ± | 0.00 ± | 0.00 ± | 0.00 ± | 0.00 ± |
| control group | 0.00 | 0.00 | 0.00 | 0.00 | 0.00 | 0.00 |
| Model control | 1.88 ± | 1.96 ± | 1.79 ± | 1.04 ± | 1.00 ± | 0.75 ± |
| group | 0.44 | 0.49 | 0.62 | 0.77 | 0.56 | 0.66 |
| C1 | 2.58 ± | 2.08 ± | 2.08 ± | 1.17 ± | 1.42 ± | 0.62 ± |
| intraperitoneal | 0.35 | 0.58 | 0.50 | 0.64 | 0.97 | 0.49 |
| low-dose group | | | | | | |
| C1 | 2.46 ± | 2.00 ± | 2.00 ± | 1.29 ± | 0.42 ± | 0.50 ± |
| intraperitoneal | 0.43 | 0.89 | 0.89 | 1.03 | 0.66 | 0.59 |
| high-dose | | | | | | |
| group | | | | | | |
| C1 vein low- | 2.00 ± | 2.04 ± | 1.92 ± | 1.04 ± | 0.87 ± | 0.33 ± |
| dose group | 0.25 | 0.28 | 0.53 | 0.74 | 0.85 | 0.44 |
| C1 vein high- | 1.88 ± | 2.04 ± | 1.83 ± | 1.13 ± | 0.92 ± | 0.50 ± |
| dose group | 0.25 | 0.28 | 0.43 | 0.75 | 0.92 | 0.40 |

Compared with the normal control group, the colon length in each group was significantly decreased, and the ulcer area was increased. The C1 intraperitoneal injection high-dose group had a tendency to improve the ulcer area, but there was no significant improvement in the colon length (Table 33).

TABLE 33
The effect of test product C1 on the colon length and ulcer area of
DNBS-induced Crohn's disease model rats (n = 6-8, Mean ± SD)

| | Dosage | Colon length | Ulcer area |
|---|---|---|---|
| Groups | (cells/only) | (cm) | (cm²) |
| Normal control | 0 | 22.5 ± 1.8 | 0.0 ± 0.0 |
| group | | | |
| Model control | 0 | 18.8 ± 0.9 | 5.7 ± 5.1 |
| group | | | |
| C1 intraperitoneal | 1 × 10⁶ | 18.0 ± 1.2 | 4.5 ± 4.5 |
| low-dose group | | | |
| C1 intraperitoneal | 1 × 10⁷ | 17.4 ± 2.2 | 1.5 ± 2.3 |
| high-dose group | | | |
| C1 vein low-dose | 1 × 10⁶ | 17.8 ± 1.1 | 2.4 ± 3.5 |
| group | | | |
| C1 vein high-dose | 1 × 10⁷ | 18.9 ± 1.4 | 4.6 ± 6.2 |
| group | | | |

Gross necropsy of the euthanized animals on D6 showed melanosis in different areas of the colon in the model control group and C1 groups, and the incidence of the model control group was 7/8; the incidence of the C1 intraperitoneal low-dose group was 5/8; the incidence of the C1 intraperitoneal high-dose group was 2/6; the incidence of the C1 intravenous low-dose group was 3/7; the incidence of the C1 intravenous high-dose group was 3/7; each administration group had a certain reduction in the incidence of gross ulcer.

Under the microscope, colonic mucosal ulcers in the model control group and C1 administration groups were seen, mainly manifested as mucosal epithelial necrosis and exudation of neutrophils in all layers of the intestinal wall (mucosa, submucosa, muscular layer, adventitia), and granulation tissue formation; mucosal hemorrhage and crypt abscess were also seen. There was no obvious improvement in the test product C1.

In conclusion, the intraperitoneal injection of the test product C1 (10⁷/only, 2 times per week) and the tail vein injection of the test product C1 (10⁶/only, 2 times per week) had a certain tendency to alleviate the colonic injury caused by modeling.

### INDUSTRIAL APPLICATION

The present invention adopts a serum-free medium cell culture process in the working bank and preparation stage of the preparation of mesenchymal stem cells, which avoids the possible exogenous virus contamination issues caused by animal-derived raw materials, while the composition and content of the serum-free medium are relatively clear, which avoids the influence of differences between serum batches on product quality, and further improves the safety and stability of the stem cell preparation. In addition, in order to ensure the timeliness, availability, safety, and effectiveness of clinical use, the present invention adopts the cryopreservation mode, and develops the preparation into a cryopreservation preparation form prepared in advance as an “off-the-shelf” product. Compared with fresh preparations, cryopreservation preparations are more convenient to store, and can be directly administered intravenously after thawing without changing the liquid, avoiding possible contamination risk during dispensing, and is more conducive to clinical use. Moreover, the cryopreservation preparation has a long shelf life, and multiple tests for the function and safety of the preparation can be completed during the shelf life, with lower safety risks, which can effectively improve clinical benefits.

What is claimed is:

1-20. (canceled)

21. A method for preventing and/or treating a disease or condition in a subject, wherein the method comprises the following steps: administering to the subject a prophylactically or therapeutically effective amount of human umbilical cord-derived mesenchymal stem cells, wherein the subject suffers from a disorder selected from the group consisting of the following:
N1) diseases caused by novel coronavirus;
N2) idiopathic pulmonary interstitial fibrosis;
N3) acute lung injury;
N4) liver fibrosis; and
N5) Crohn's disease.

22. The method according to claim 21, wherein the human umbilical cord-derived mesenchymal stem cells are prepared and obtained by the following steps:
(1) culturing an isolated umbilical cord tissue block, and obtaining P0 mesenchymal stem cells when the cell confluence reaches 40%-60% after the mesenchymal stem cells migrating out of the umbilical cord tissue block;
(2) after completion of step (1), subculturing the P0 mesenchymal stem cells until the cell confluence is 60%-80% to obtain P1 mesenchymal stem cells;
(3) after completion of step (2), subculturing the P1 mesenchymal stem cells until the cell confluence is 60%-80% to obtain P2 mesenchymal stem cells;
(4) after completion of step (3), performing cell cryopreservation on the P2 mesenchymal stem cells to obtain cryopreserved P2 seed bank cells;
(5) after completion of step (4), thawing the cryopreserved P2 seed bank cells and performing subculturing until the cell confluence is 60%-80% to obtain P3 mesenchymal stem cells;
(6) after completion of step (5), subculturing the P3 mesenchymal stem cells until the cell confluence is 60%-80% to obtain P4 mesenchymal stem cells, and the P4 mesenchymal stem cells are used for the administration to the subjects.

23. The method according to claim 22, wherein it further comprises the following steps:
(7) performing cell cryopreservation on the P4 mesenchymal stem cells to obtain cryopreserved P4 working bank cells;
(8) after completion of step (7), thawing the cryopreserved P4 working bank cells and performing subculturing until the cell confluence is 60-80% to obtain P5 mesenchymal stem cells; and
(9) after completion of step (8), the P5 cells are sequentially digested, washed and resuspended to obtain the human umbilical cord-derived mesenchymal stem cells for the administration to the subject.

24. The method according to claim 21, wherein the disorders caused by novel coronavirus is selected from one or more of the following: A. increased viral load in the lungs; B. the inflammatory infiltration around small blood vessels; C. the inflammatory infiltration around bronchioles; and D. lung bronchiole epithelial cell degeneration.

25. The method according to claim 22, wherein in the step (1), step (2) and step (3), fetal bovine serum complete medium is used for cell culture; or in the step (4), the cell cryopreservation method is to resuspend the cells with a cryopreservation solution A to obtain a cell suspension; the cryopreservation solution A is composed of fetal bovine serum and dimethyl sulfoxide; or in the step (5) and step (6), the serum-free complete medium is used for cell culture.

26. The method according to claim 25, wherein a concentration of the cell suspension is 3×10⁶ cells/mL.

27. The method according to claim 25, wherein a volume ratio of the fetal bovine serum to the dimethyl sulfoxide is 9:1.

28. A kind of isolated human umbilical cord-derived mesenchymal stem cells, prepared and obtained by the following methods:
(1) culturing an isolated umbilical cord tissue block, and obtaining P0 mesenchymal stem cells when the cell confluence reaches 40%-60% after the mesenchymal stem cells migrating out of the umbilical cord tissue block;
(2) after completion of step (1), subculturing the P0 mesenchymal stem cells until the cell confluence is 60%-80% to obtain P1 mesenchymal stem cells;
(3) after completion of step (2), subculturing the P1 mesenchymal stem cells until the cell confluence is 60%-80% to obtain P2 mesenchymal stem cells;
(4) after completion of step (3), performing cell cryopreservation on the P2 mesenchymal stem cells to obtain cryopreserved P2 seed bank cells;
(5) after completion of step (4), thawing the cryopreserved P2 seed bank cells and performing subculturing until the cell confluence is 60%-80% to obtain P3 mesenchymal stem cells;
(6) after completion of step (5), subculturing the P3 mesenchymal stem cells until the cell confluence is 60%-80% to obtain P4 mesenchymal stem cells;
(7) after completion of step (6), performing cell cryopreservation on the P4 mesenchymal stem cells to obtain cryopreserved P4 working bank cells;
(8) after completion of step (7), thawing the cryopreserved P4 working bank cells and performing subculturing until the cell confluence is 60-80% to obtain P5 mesenchymal stem cells; and
(9) after completion of step (8), the P5 cells are sequentially digested, washed and resuspended to obtain the human umbilical cord-derived mesenchymal stem cells.

29. The isolated human umbilical cord-derived mesenchymal stem cells according to claim 28, wherein in the step (7), the cell cryopreservation method is to resuspend the cells with a cryopreservation solution B to obtain a cell suspension; the cryopreservation solution B is composed of serum-free medium base, dimethyl sulfoxide and human albumin solution; or in step (8), the serum-free complete medium is used for cell culture.

30. The isolated human umbilical cord-derived mesenchymal stem cells according to claim 29, wherein the concentration of the cell suspension is 8× 10⁶ cells/mL.

31. The isolated human umbilical cord-derived mesenchymal stem cells according to claim 29, wherein a volume ratio of the serum-free medium base, the dimethyl sulfoxide and the human albumin solution is 7:2:1.

32. A cryopreservation preparation of human umbilical cord-derived mesenchymal stem cells, which comprises the isolated human umbilical cord-derived mesenchymal stem cells according to claim 28 and a cell cryoprotectant.

33. The cryopreservation preparation of human umbilical cord-derived mesenchymal stem cells according to claim 32, wherein the cell cryoprotectant is selected from the group consisting of the following: a multiple electrolyte solution, dimethyl sulfoxide, a dextran 40 sodium chloride injection and a human albumin solution.

34. The cryopreservation preparation of human umbilical cord-derived mesenchymal stem cells according to claim 32 for use in the prevention or treatment of a disorder in a subject selected from the group consisting of the following:
N1) diseases caused by novel coronavirus;
N2) idiopathic pulmonary interstitial fibrosis;
N3) acute lung injury;
N4) liver fibrosis; and
N5) Crohn's disease.

35. The cryopreservation preparation of human umbilical cord-derived mesenchymal stem cells according to claim 34, wherein the disorders caused by novel coronavirus is selected from one or more of the following: A. increased viral load in the lungs; B. the inflammatory infiltration around small blood vessels; C. the inflammatory infiltration around bronchioles; and D. lung bronchiole epithelial cell degeneration.

36. A method for preparing a cryopreservation preparation of human umbilical cord-derived mesenchymal stem cells comprising following steps: resuspending the isolated human umbilical cord-derived mesenchymal stem cells according to claim 28 with a cell cryoprotectant, and then lowering the temperature of the obtained cell suspension to obtain the cryopreservation preparation of human umbilical cord-derived mesenchymal stem cells.

37. The method according to claim 36, wherein the cell cryoprotectant is selected from the group consisting of the following: a multiple electrolyte solution, dimethyl sulfoxide, a dextran 40 sodium chloride injection and a human albumin solution.

38. The method according to claim 37, wherein a volume ratio of the multiple electrolyte solution, the dimethyl sulfoxide, the dextran 40 sodium chloride injection and the human albumin solution is (70-80):(5-10):(5-10):(5-10) or 70:(5-10):(5-10):(5-10) or 80:(5-10):(5-10): (5-10) or (70-80):5:(5-10):(5-10) or (70-80):10:(5-10):(5-10) or (70-80):(5-10):5:(5-10) or (70-80):(5-10):10:(5-10) or (70-80):(5-10):(5-10):5 or (70-80):(5-10):(5-10):10.

39. The method according to claim 36, wherein a concentration of the human umbilical cord-derived mesenchymal stem cells in the cryopreservation preparation of human umbilical cord-derived mesenchymal stem cells is 2.5×10⁶-1×10⁷ cells/mL.

40. The method according to claim 36, wherein the procedure of the lowering the temperature comprises the following steps:
Step 1: initial temperature to 4° C.;
Step 2: 1° C./min down to −5° C.;
Step 3: 20° C./min until the cavity temperature is −40° C.;
Step 4: 10° C./min until the cavity temperature is −20° C.;
Step 5: 2° C./min until the sample drops to −40° C.;
Step 6: 10° C./min until the sample drops to −80° C.; and
Step 7: end.


SEQUENCE LISTING (6 sequences)

SEQ ID NO: 1 - DNA, 23 bp, synthetic construct
  source 1..23: mol_type = other DNA
ggtgagaagagatgtctgaatcc

SEQ ID NO: 2 - DNA, 22 bp, synthetic construct
  source 1..22: mol_type = other DNA
gtccatccttggaagcactgca

SEQ ID NO: 3 - DNA, 22 bp, synthetic construct
  source 1..22: mol_type = other DNA
gctgtaaggacatcgcctacca

SEQ ID NO: 4 - DNA, 23 bp, synthetic construct
  source 1..23: mol_type = other DNA
agaatcaccagcagcaagtgtcc

SEQ ID NO: 5 - DNA, 22 bp, synthetic construct
  source 1..22: mol_type = other DNA
gcctgatctcatagagtctggc

SEQ ID NO: 6 - DNA, 22 bp, synthetic construct
  source 1..22: mol_type = other DNA
tgcatcccagaactagacgtgc